(19) United States
(12) Patent Application Publication
(10) Pub. No.: US 2010/0183620 A1
(43) Pub. Date: Jul. 22, 2010

(54) COMPOSITIONS AND METHODS FOR REGULATING COLLAGEN AND SMOOTH MUSCLE ACTIN EXPRESSION BY SERPINE2

(76) Inventors: KAUMUDI BHAWE, Palo Alto, CA (US); Elizabeth Bosch, Cupertino, CA (US); Kathleen Boyle, Alameda, CA (US); Arthur Brace, Redwood City, CA (US); Anuk Das, Berwyn, PA (US); Francis Farrell, Doylestown, PA (US); Pitchumani Sivakumar, King of Prussia, PA (US); Jeffrey Finer, Foster City, CA (US); Kristen Pierce, Burlingame, CA (US); Kathleen M. Sullivan, Oakland, CA (US); Brian Wong, Los Altos, CA (US)

Correspondence Address: LAW OFFICE OF SALVATORE ARRIGO, 1050 CONNECTICUT AVE. NW, 10TH FLOOR, WASHINGTON, DC, 20036, US

(21) Appl. No.: 12/626,534

(22) Filed: Nov. 25, 2009

Related U.S. Application Data
(60) Provisional application No. 61/118,180, filed on Nov. 26, 2008.

(51) Int. Cl. A61K 39/395; A61K 31/7088; A61K 38/02; A61K 38/16; A61P 21/00; C12Q 1/68

(52) U.S. Cl. 424/141.1; 424/130.1; 514/44 A; 514/2; 514/12; 435/6

(57) ABSTRACT

The invention encompasses methods and compositions for increasing or decreasing collagen 1A1 expression and/or α-smooth muscle actin expression in lung fibroblasts using SERPINE2 and antagonists of SERPINE2. The invention also encompasses methods and compositions for increasing or decreasing the formation of myofibroblasts. The invention further provides methods and compositions for treatment of lung diseases, such as idiopathic pulmonary fibrosis and chronic obstructive pulmonary disease.

### CROSS-REFERENCE TO RELATED APPLICATIONS

This application claims the benefit of U.S. Provisional Application No. 61/118,180, filed Nov. 26, 2008, which is incorporated herein by reference.

### BACKGROUND OF THE INVENTION

There are many different types of lung diseases involving lung fibrosis, such as idiopathic pulmonary fibrosis (IPF), acute lung injury (ALI), acute respiratory distress syndrome (ARDS), asthma, and chronic obstructive pulmonary disease (COPD). Howell et al., Am. J. Path. 159:1383-1395 (2001), U.S. Patent Publ. No. 2009/0136500 A1.

For example, idiopathic pulmonary fibrosis (IPF) is a common form of interstitial lung disease that is characterized by fibroblast proliferation and excessive collagen deposition. Hardie et al., Am. J. of Respir. Cell Mol. Biol. 327:309-321 (2007). IPF may be the result of a chronic inflammatory process that initiates focal accumulation of extracellular matrix in the interstitium. Alternatively, IPF may be caused by pulmonary epithelial injury may lead to abnormal wound healing with excessive extracellular matrix formation. To date, there is no effective treatment for IPF. Hardie et al., (2007); Meltzer et al., Orphanet Journal of Rare Diseases, 3:8 (2008).

Pulmonary fibroblast to myofibroblast conversion is a pathophysiological feature of idiopathic pulmonary fibrosis and other pulmonary diseases, such as chronic obstructive pulmonary disease (COPD). Dunkern et al., Eur J. Pharmacol. 572(1):12-22 (2007).

Reduced levels of antifibrinolytic activity have been reported in the alveolar fluids of IPF patients. Chapman et al., Am. Rev. Respir. Dis. 133:437-443 (1986). The levels of plasminogen activator inhibitor (PAI-1) antigen (also known as SERPINE1) in lung fluids and levels of PAI-2 antigen (also known as SERPINB2) in lung cell lysates were reported to be higher in patients than in normal subjects. Id. PAI-1 is involved in pulmonary fibrosis. Gharee-Kermani et al., Expert Opin. Investig. Drugs 17:905-916, 2008. Urokinase plasminogen activator (uPA) is the major activator of fibrinolysis in extravascular tissue. Id. PAI-1 inhibits uPA. Id. Thus, the proteolytic properties of the plasminogen system may play an important role in the modulation of lung repair and fibrosis. Id.

SERPINE2 is an irreversible extracellular serine proteinase inhibitor. It is overexpressed in cancers of the pancreas, colon, and stomach. Neesse et al., Pancreatology 7:380-385, 2007. SERPINE2 is also known as protease nexin I (PN-1) and glia-derived nexin (GDN). SERPINE2 also inhibits extracellular urokinase plasminogen activator. Scott et al., J. Biol. Chem. 258:4397-4403, 1983.

Transfection of a pancreatic cancer cell line with SERPINE2 caused an enhancement of the local invasiveness of xenograft tumors, accompanied by a massive increase in extracellular matrix (ECM) production in the invasive tumors. Buchholz et al., Cancer Research 63:4945-4951 (2003). The ECM deposits were positive for type I collagen, fibronectin, and laminin. Id.

SERPINE2 protein has been found to be expressed in mouse and human lungs. DeMeo et al., Am. J. Hum. Gen. 78:253-264 (2006). This article suggested that overexpression of SERPINE2 was associated with Chronic Obstructive Pulmonary Disease. SERPINE2 has been demonstrated to be an extracellular inhibitor of trypsin-like serine proteases, such as thrombin, trypsin, plasmin, and urokinase. Id.

SERPINE2 is secreted by fibroblasts. Farrell et al., J. Cell Physiol. 134:179-188, 1988. SERPINE2 forms complexes with certain serine proteases in the extracellular environment including thrombin, urokinase, and plasmin, which are then internalized by cells and degraded. Id. SERPINE2 is present on the surface of fibroblasts, bound to the extracellular matrix. Id.

Fibroblasts isolated from skin lesions of scleroderma patients overexpress collagens and other matrix components. Strehlow et al., J. Clin. Invest. 103:1179-1190 (1999). SERPINE2 was overexpressed in scleroderma fibroblasts. Id. Transient or stable expression of SERPINE2 in mouse 3T3 fibroblasts increased collagen α-1(I) promoter activity or endogenous collagen transcript levels, respectively. Id. SERPINE2 mutagenized at its active site failed to increase collagen promoter activity. Id. Overexpression of SERPINE2 in the antisense orientation appeared to inhibit expression from the collagen promoter in mouse 3T3 fibroblasts. Id. In Strehlow et al., 1999, Human SERPINE2 point mutations, R364K and S365T, were made and confirmed to lack formation of higher order complexes with thrombin.

The low density lipoprotein receptor-related protein (LRP) is a receptor responsible for the internalization of protease-SERPINE2 complexes. Knauer et al., J. Biol. Chem. 272: 29039-29045, 1997. Binding of Thrombin-SERPINE2 to LRP is mediated by amino acids 47-58. Knauer et al., J. Biol. Chem. 272:12261-12264, 1997. SERPINE2 point mutations in the LRP binding region, H48A, and double mutant H48A and D49A had similar thrombin complex formation rates similar to wild type but had reduced catabolism and internalization down to 50% and 15% of wild type. Knauer et al., J. Biol. Chem. 274:275-281, 1999.

The primary effector cell in IPF is the myofibroblast. Scotton and Chambers, Chest 132:1311-1321 (2007). Myofibroblast cells are highly synthetic for collagen, have a contractile phenotype, and are characterized by the presence of α-smooth muscle actin stress fibers. Id. Myofibroblasts may be derived by activation/proliferation of resident lung fibroblasts, epithelial-mesenchymal differentiation, or recruitment of circulating fibroblastic stem cells (fibrocytes). Id. Myofibroblasts are involved in the wound healing process. Hinz et al., Am. J. Pathology 170:1807-1816 (2007). Transforming growth factor (TGF) (31 has been shown to be involved in inducing the generation of myofibroblasts. Id.

In one study of patients with pulmonary fibrosis, a marked increase in the expression of genes encoding muscle proteins, such as α-smooth muscle actin, γ-smooth muscle actin, and calponin, and integrin α7β1, was observed. Zuo et al., P.N.A.S. 99:6292-6297 (2002).

In a mouse model of pulmonary fibrosis, induction of fibrosis with TGF-α caused an increase in the lung RNA levels of several extracellular matrix proteins within 1-4 days, including procollagens type I, al (COL1A1), COL3A1, COL5A2, and COL15A1, and elastin. Hardie et al., 2007. The levels of a number of RNAs encoding defense/immunity proteins increased after TGF-α was no longer expressed, including SERPINE2. Id. It was noted that SERPINE2 was not yet associated with IPF. Id.

The rate of reaction of SERPINE2 with thrombin is increased by heparin. Wallace et al., Biochem J. (1989) 257, 191-196. The heparin-binding site of SERPINE2 has been localized by site-directed mutagenesis. Stone et al., Biochem. 33:7731-7735, 1994. The heparin binding region of SERPINE2 has been identified as amino acids 90-105. Mutation of all 7 lysine residues to glutamic acid residues eliminated heparin binding, heparin-mediated ability to accelerate thrombin complex formation, and ability of Thrombin-SERPINE1 to bind to fibroblast cell surface, as measured via degradation. Stone et al., 1994; Knauer et al., JBC 1997, 272:29039-29045, 1997.

Serpins are made up of three β-sheets and 8-9 helices. Law et al., Genome Biology 7:216, 2006. The reactive center loop (RCL) interacts with target proteases. Id. The cleavage of the serpin results in a conformational change that distorts the active site of the protease, which prevents efficient hydrolysis of the acyl intermediate and subsequent release of the protease. Id. Thus, serpins are irreversible, suicide inhibitors. Id.

Many different types of antagonists of serpins have been generated. For example, monoclonal antibodies against SERPINE2 can block its inhibition of target proteases. Wagner et al., Biochemistry 27: 2173-2176, 1988; Boulaftali et al. Blood First Edition Paper, prepublished online Oct. 23, 2009; DOI 10.1182/blood-2009-04-217240. Similarly, neutralizing antibodies, including scFV fragments, against SERPINE1 (i.e., plasminogen activator inhibitor-1) have been made. See, e.g., Verbeke et al., J. Thromb. Haemost. 2:298-305, 2004, and Brooks et al., Clinical & Experimental Metastasis 18:445-453, 2001. Antisense RNAs and oligonucleotides have also been used to inhibit SERPINE2 and SERPINE1 expression. Kim and Loh, Mol. Biol. Cell. 17:789-798, 2006, and Sawa et al., J. Biol. Chem. 269:14149-14152, 1994.

RNA interference has also been used to suppress SERPINE1 expression. Kortlever et al., Nature Cell Biology 8:877-884, 2006. Inactivation of SERPINE1 was also successful using a 14 amino acid peptide corresponding to the reactive center loop of SERPINE1. Eitzman et al., J. Cin. Invest. 95:2416-2420, 1995. Other serpins have been likewise inhibited by peptides corresponding to the reactive center loop. Bjork et al., J. Biol. Chem. 267:1976-1982, 1992; Schulze et al., Eur. J. Biochem. 194:51-56, 1990. A low molecular weight molecule, XR5967, which is a diketopiperazine, has also been shown to inhibit SERPINE1 activity. Brooks et al., Anticancer Drugs 15:37-44, 2004.

There are many fibrotic lung disease involving lung fibroblasts. For example, idiopathic pulmonary fibrosis is a chronic, progressive, and frequently fatal interstitial lung disease for which there are no proven drug therapies. Gharaee-Kermani et al., 2008. Thus, a need exists for additional compositions and methods for treating fibrotic lung diseases involving lung fibroblasts, such as IPF and COPD.

### SUMMARY OF THE INVENTION

It has been found that the administration of purified SERPINE2 to human lung fibroblast cells results in increased expression of collagen 1A1 and α-smooth muscle actin. Administration of a SERPINE2 LRP binding mutant, lacking the ability to bind the low density lipoprotein receptor-related protein (LRP), also resulted in increased expression of collagen 1A1 and α-smooth muscle actin. Administration of a, and a SERPINE2 protease interaction mutant, lacking the ability to interact with its target proteases, showed no ability to increase expression of collagen 1A1 and α-smooth muscle actin expression. Administration of a SERPINE2 protease inhibition mutant, that should retain the ability to interact with its target proteases, but that not fully block the activity of the proteases (Strehlow et al., 1999), resulted in an intermediate level of expression of collagen 1A1 and α-smooth muscle actin.

Administration of polyclonal antibodies against SERPINE2 abolished the SERPINE2-induced increase in collagen 1A1 in a dose-dependent manner. In addition, TGF-β induced a large increase in SERPINE2 mRNA expression in normal human lung fibroblasts, and treatment of mice with bleomycin caused an increase in the levels of SERPINE2 protein expression in lung lysates.

These results indicate that SERPINE2 can cause an increase in formation of activated myofibroblasts with increased expression of collagen 1A1 and α-smooth muscle actin, as seen in idiopathic pulmonary fibrosis. The invention encompasses methods and compositions for increasing collagen 1A1 expression and/or increasing α-smooth muscle actin expression in lung fibroblasts. For example, recombinant SERPINE2 can be added to lung fibroblast cells to increase collagen 1A1 and α-smooth muscle actin expression and myofibroblast formation. Since SERPINE2 is an extracellular protease inhibitor, it can be produced by the lung fibroblasts themselves or come from another source, such as added protein or production by neighboring cells. These compositions and methods are useful for increasing the expression of collagen 1A1 and/or α-smooth muscle actin in lung fibroblasts and in drug screening assays for antagonists of SERPINE2. These compositions and methods are also useful for drug assays for compositions that antagonize fibrotic activity in vivo. For example, mice can be administered purified SERPINE2, together with other compounds, and used to screen for compounds that antagonize fibrosis. The compositions and methods of the invention are also useful for increasing the formation of activated myofibroblasts to help in wound healing.

In various embodiments, the invention encompasses methods for increasing the level of collagen 1A1 production and/or α-smooth muscle actin production in a human lung fibroblast cell comprising administering SERPINE2 to a cell and detecting an increase in collagen 1A1 and/or α-smooth muscle actin expression in the human lung fibroblast cell. In preferred embodiments, the SERPINE2 is administered in an expression vector or as a purified protein. Preferably, the increase in collagen expression is detected by measuring an increase in the level of collagen 1A1 RNA and/or by measuring an increase in the level of α-smooth muscle actin RNA production.

Since exposure of human lung fibroblasts to elevated levels of SERPINE2 causes increased expression of collagen 1A1 and α-smooth muscle actin, which is blocked by interfering with the ability of SERPINE2 to bind to its protease target, an antagonist of SERPINE2 can cause a decrease in collagen 1A1 and α-smooth muscle actin expression in human lung fibroblast cells exposed to elevated levels of SERPINE2. In this way, an antagonist of SERPINE2 can block the effects of exposing human lung fibroblast cells to elevated levels of SERPINE2, such as the generation of myofibroblasts. Thus, the invention encompasses methods and compositions for decreasing collagen 1A1 expression and/or decreasing α-smooth muscle actin expression in lung fibroblasts using antagonists of SERPINE2. Such antagonists are useful in decreasing collagen 1A1 and/or α-smooth muscle actin expression in lung fibroblasts and in preventing fibrosis mediated by lung fibroblasts, such as by the action of myofibroblasts.

In various embodiments, the invention encompasses methods for inhibiting the level of collagen 1A1 and/or α-smooth muscle actin expression in a human lung fibroblast cell exposed to an elevated level of SERPINE2 comprising administering an antagonist of SERPINE2 to the human lung fibroblast cell. In one embodiment, the method comprises detecting a decrease in collagen 1A1 and/or α-smooth muscle actin expression in the lung fibroblast cell. In various embodiments, the lung fibroblast cell is exposed to TGF-β prior to exposure to the antagonist. In some embodiments, the lung fibroblast cell is exposed to IL-13 prior to exposure to the antagonist. Preferably, the antagonist of SERPINE2 is an antibody, an RNAi molecule, an antisense nucleic acid molecule, a peptide, or a small molecule inhibitor of SERPINE2.

The antagonist of SERPINE2 can also be used in combination with other inhibitors of pulmonary fibrosis, including antagonists of SERPINE1, such as antibodies, etc.

The invention also encompasses methods for inhibiting the formation of myofibroblasts from human lung fibroblast cells exposed to an elevated level of SERPINE2 comprising administering an antagonist of SERPINE2 to the human lung fibroblast cells. In various embodiments, the lung fibroblast cells are exposed to TGF-β prior to exposure to the antagonist. In some embodiments, the lung fibroblast cells are exposed to IL-13 prior to exposure to the antagonist. Preferably, the antagonist of SERPINE2 is an antibody, an RNAi molecule, an antisense nucleic acid molecule, a peptide, or a small molecule inhibitor of SERPINE2.

The invention further encompasses methods for inhibiting the level of collagen 1A1 and/or α-smooth muscle actin expression in a human lung fibroblast cell exposed to SERPINE2 comprising administering an antagonist of SERPINE2 to the human lung fibroblast cell. In one embodiment, the method comprises detecting a decrease in collagen 1A1 and/or α-smooth muscle actin expression in the lung fibroblast cell. In various embodiments, the lung fibroblast cell is exposed to TGF-β prior to exposure to the antagonist. In some embodiments, the lung fibroblast cell is exposed to IL-13 prior to exposure to the antagonist. Preferably, the antagonist of SERPINE2 is an antibody, an RNAi molecule, an antisense nucleic acid molecule, a peptide, or a small molecule inhibitor of SERPINE2.

In the context of this invention, antagonists of SERPINE2 include any molecule(s) that can specifically inhibit the RNA expression, protein expression, or protein activity of SERPINE2. Thus, antagonists of SERPINE2 include antibodies which specifically bind to SERPINE2 and inhibit its biological activity; antisense nucleic acids RNAs that interfere with the expression of SERPINE2; small interfering RNAs that interfere with the expression of SERPINE2; small peptide inhibitors of SERPINE2, and small molecule inhibitors of SERPINE2. For example, an antagonist antibody that specifically binds to SERPINE2 and blocks its biological activity can be added to lung fibroblast cells exposed to elevated levels of SERPINE2 to decrease collagen 1A1 and α-smooth muscle actin expression. Similarly, an antagonist antibody that specifically binds to SERPINE2 can be added to lung fibroblast cells exposed to elevated levels of SERPINE2 to decrease the formation of myofibroblasts.

The effects of elevated SERPINE2 levels on increasing collagen 1A1 and α-smooth muscle actin expression indicated to the inventors that exposure of human lung fibroblasts to elevated levels of SERPINE2 promoted the formation of myofibroblasts, which are the primary effector cells involved various lung diseases, including IPF and COPD. Thus, the invention encompasses methods and compositions for decreasing collagen 1A1 expression and/or decreasing α-smooth muscle actin expression in lung fibroblasts in patients with overexpression of collagen 1A1 expression and/or α-smooth muscle actin, such as IPF and COPD patients, by administering an antagonist of SERPINE2 to lung fibroblasts, and by decreasing myofibroblast formation.

The invention includes the use of an antagonist of SERPINE2 for the preparation of a medicament for the treatment of a medical condition, wherein the medical condition is lung fibrosis, especially one in which human lung fibroblast cells are exposed to an elevated level of SERPINE2. In preferred embodiments, the medical condition is idiopathic pulmonary fibrosis (IPF) or chronic obstructive pulmonary disease (COPD). Preferably, the antagonist of SERPINE2 is an antibody, e.g., a monoclonal antibody. In various embodiments, the antagonist of SERPINE2 is an RNAi molecule, an antisense nucleic acid molecule, a peptide, or a small molecule inhibitor of SERPINE2. The antagonist of SERPINE2 can also be used in combination with other inhibitors of pulmonary fibrosis, including antagonists of SERPINE1, such as antibodies, etc.

In this way, the invention provides methods and compositions for treatment of lung diseases, such as IPF, ALI, ARDS, asthma, and COPD. Such compositions can be provided prophylactically or therapeutically to patients having or at risk of having symptoms of such diseases.

### BRIEF DESCRIPTION OF THE DRAWINGS

The invention is more fully understood with reference to the drawings in which:

FIG. 1 depicts the results of an assay for the effect of purified SERPINE2 protein on human lung fibroblasts. A bDNA assay was performed on normal human lung fibroblasts treated with purified SERPINE2 at a concentration of 0, 2.5, or 5 μg/ml with 0.5 ng/ml of TGF-β for 48 hours. β-actin (ACTB), α-smooth muscle actin (ACTA2), and collagen 1A1 (COL1A1) RNA levels were measured.

FIG. 2 depicts the results of an assay for the effect of protein supernatants from cells transfected with wild-type (WT) SERPINE2, a SERPINE2 LRP binding mutant, SERPINE2 protease inhibition mutant, and a SERPINE2 protease interaction mutant on β-actin (ACTB), α-smooth muscle actin (ACTA2), and collagen 1A1 (COL1A1) RNA levels. A supernatant from a vector control (VCM) was also used. A bDNA assay was performed on normal human lung fibroblasts treated with the SERPINE2-containing or VCM supernatants and with 0.05 ng/ml of TGF-β for 48 hours.

FIG. 3 depicts the results of an assay for the effect of protein supernatants from cells transfected with wild-type (WT) SERPINE2, a SERPINE2 LRP binding mutant, SERPINE2 protease inhibition mutant, and a SERPINE2 protease interaction mutant on β-actin (ACTB), α-smooth muscle actin (ACTA2), and collagen 1A1 (COL1A1) RNA levels. A supernatant from a vector control (VCM) was also used. A bDNA assay was performed on normal human lung fibroblasts treated with the SERPINE2-containing or VCM supernatants and with 0.5 ng/ml of TGF-β for 48 hours.

FIGS. 4A and B depict the induction of collagen protein production by increasing concentrations of SERPINE2 protein in the presence of two different concentrations of TGF-β.

FIG. 5 depicts the induction of SERPINE2 RNA production by TGF-β in NHLF cells.

FIG. 6 depicts the inhibition of mouse SERPINE2 induced collagen production in lung fibroblasts using a polyclonal antibody to mouse SERPINE2. ### p<0.001 compared to no treatment, *** p<0.001 compared to 0.5 ng/ml of TGFβ, one way ANOVA and Newman Keuls.

FIG. 7 depicts SERPINE2 protein levels in lung lysates of mice treated with saline or bleomycin for 7 or 14 days. Statistical significance was determined using One way ANOVA with Tukey's Post test. SERPINE2 levels (51 KD band) are significantly increased in Bleo-treated lung lysates have increased SERPINE2 protein, *** p<0.0001 compared to saline-treated mouse lungs.

### DETAILED DESCRIPTION OF THE INVENTION

The invention encompasses methods and compositions for increasing collagen 1A1 expression and/or increasing α-smooth muscle actin expression in lung fibroblasts using SERPINE2.

The invention further encompasses methods and compositions for decreasing collagen 1A1 expression and/or decreasing α-smooth muscle actin expression in lung fibroblasts using antagonists of SERPINE2. An antagonist of SERPINE2 can be added to lung fibroblast cells exposed to elevated levels of SERPINE2 to decrease collagen 1A1 and α-smooth muscle actin expression. Similarly, an antagonist of SERPINE2 can be added to lung fibroblast cells exposed to elevated levels of SERPINE2 to decrease the formation of myofibroblasts.

Exposure of lung fibroblast cells to SERPINE2 can be inhibited by administration of an antagonist of SERPINE2. The antagonist can reduce or block the RNA expression, protein expression, or protein activity of SERPINE2.

An “elevated” level of SERPINE2 refers to a level of SERPINE2 protein that exceeds the average value for the cells and/or tissue. For example, addition of SERPINE2 to a culture of lung fibroblast cells results in an elevated level of SERPINE2. Also, levels of SERPINE2 in the bronchial lavage of patients that exceed the average values of SERPINE2 for bronchial lavage samples are elevated.

Exposure of lung fibroblast cells to elevated level of SERPINE2 can be inhibited by administration of an antagonist of SERPINE2. The antagonist can reduce or block the RNA expression, protein expression, or protein activity of SERPINE2.

The invention encompasses methods and compositions for decreasing collagen 1A1 expression and/or decreasing α-smooth muscle actin expression in lung fibroblasts in IPF patients by administering an antagonist of SERPINE2 to lung fibroblasts, and by decreasing myofibroblast formation. In this way, the invention provides methods and compositions for treatment of idiopathic pulmonary fibrosis.

### Nucleic Acid Molecules

In one embodiment, the invention relates to certain isolated SERPINE2 nucleotide sequences that are free from contaminating endogenous material. A “nucleotide sequence” refers to a polynucleotide molecule in the form of a separate fragment or as a component of a larger nucleic acid construct. The nucleic acid molecule has been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

SERPINE2 nucleic acid molecules include DNA in both single-stranded and double-stranded form, as well as the RNA complement thereof. DNA includes, for example, cDNA, genomic DNA, chemically synthesized DNA, DNA amplified by PCR, and combinations thereof. The DNA molecules of the invention include full length genes encoding SERPINE2 as well as polynucleotides and fragments thereof. The nucleic acids of the invention are normally derived from human sources, but the invention includes those derived from other sources as well.

Particularly preferred nucleotide sequences of the invention are the human sequence of SERPINE2 set forth in SEQ ID NO:1. The sequence of amino acids encoded by the DNA of SEQ ID NO:1 is shown in SEQ ID NO:2.

Due to the known degeneracy of the genetic code, wherein more than one codon can encode the same amino acid, a DNA sequence can vary from that shown in SEQ ID NO:1 and still encode a polypeptide having the amino acid sequence of SEQ ID NO:2. Such variant DNA sequences can result from silent mutations (e.g., occurring during PCR amplification), or can be the product of deliberate mutagenesis of a native sequence.

The invention thus encompasses isolated DNA sequences encoding SERPINE2 polypeptides, selected from: (a) DNA comprising the nucleotide sequence of SEQ ID NO:1; (b) DNA encoding the polypeptides of SEQ ID NO:2; (c) DNA capable of hybridization to a DNA of (a) or (b) under conditions of moderate stringency and which encodes SERPINE2 or a fragment thereof; (d) DNA capable of hybridization to a DNA of (a) or (b) under conditions of high stringency and which encodes SERPINE2 or a fragment thereof, and (e) DNA which is degenerate as a result of the genetic code to a DNA defined in (a), (b), (c), or (d) and which encode SERPINE2 or a fragment thereof. Of course, the polypeptides encoded by such DNA sequences are encompassed by the invention.

The invention thus provides equivalent isolated DNA sequences encoding biologically active SERPINE2 polypeptides selected from: (a) DNA derived from the coding region of a native mammalian SERPINE2 gene; (b) DNA of SEQ ID NO:1 or a fragment thereof, (c) DNA capable of hybridization to a DNA of (a) or (b) under conditions of moderate stringency and which encodes biologically active SERPINE2 polypeptides; and (d) DNA that is degenerate as a result of the genetic code to a DNA defined in (a), (b) or (c), and which encodes biologically active SERPINE2 polypeptides. SERPINE2 polypeptides encoded by such DNA equivalent sequences are encompassed by the invention. SERPINE2 polypeptides encoded by DNA derived from other mammalian species, wherein the DNA will hybridize to the complement of the DNA of SEQ ID NO:1, are also encompassed.

As used herein, “conditions of “moderate stringency” means use of a prewashing solution for the nitrocellulose filters 5×SSC, 0.5% SDS, 1.0 mM EDTA (pH 8.0), hybridization conditions of about 50% formamide, 6×SSC at about 42° C. (or other similar hybridization solution, such as Stark's solution, in about 50% formamide at about 42° C.), and washing conditions of about 60° C., 0.5×SSC, 0.1% SDS. “Conditions of high stringency” means hybridization conditions as above, with washing at approximately 68° C., 0.2×SSC, 0.1% SDS.

Also included as an embodiment of the invention is DNA encoding SERPINE2 polypeptide fragments and polypeptides comprising conservative amino acid substitution(s), as described below.

In another embodiment, the nucleic acid molecules of the invention also comprise nucleotide sequences that are at least 80% identical to a native SERPINE2 sequence. Also contemplated are embodiments in which a nucleic acid molecule comprises a sequence that is at least 90% identical, at least 95% identical, at least 98% identical, at least 99% identical, or at least 99.9% identical to a native SERPINE2 sequence.

As used herein, the percent identity of two nucleic acid sequences can be determined by comparing sequence information using the GAP computer program, version 6.0 described by Devereux et al. (Nucl. Acids Res. 12:387, 1984) and available from the University of Wisconsin Genetics Computer Group (UWGCG), using the default parameters for the GAP program including: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

The invention also provides isolated nucleic acids useful in the production of polypeptides. Such polypeptides can be prepared by any of a number of conventional techniques. A DNA sequence encoding SERPINE2, or desired fragment thereof, can be subcloned into an expression vector for production of the polypeptide or fragment. The DNA sequence advantageously is fused to a sequence encoding a suitable leader or signal peptide. Alternatively, the desired fragment can be chemically synthesized using known techniques. DNA fragments also can be produced by restriction endonuclease digestion of a full length cloned DNA sequence, and isolated by electrophoresis on agarose gels. If necessary, oligonucleotides that reconstruct the 5′ or 3′ terminus to a desired point can be ligated to a DNA fragment generated by restriction enzyme digestion. Such oligonucleotides can additionally contain a restriction endonuclease cleavage site upstream of the desired coding sequence, and position an initiation codon (ATG) at the N-terminus of the coding sequence.

The well-known polymerase chain reaction (PCR) procedure also can be employed to isolate and amplify a DNA sequence encoding a desired protein fragment. Oligonucleotides that define the desired termini of the DNA fragment are employed as 5′ and 3′ primers. The oligonucleotides can additionally contain recognition sites for restriction endonucleases, to facilitate insertion of the amplified DNA fragment into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, innis et al., eds., Academic Press, Inc. (1990).

### Polypeptides and Fragments Thereof

The invention encompasses polypeptides and fragments thereof in various forms, including those that are naturally occurring or produced through various techniques such as procedures involving recombinant DNA technology. For example, DNAs encoding SERPINE2 polypeptides can be derived from SEQ ID NO:1 by in vitro mutagenesis, which includes site-directed mutagenesis, random mutagenesis, and in vitro nucleic acid synthesis. Such forms include, but are not limited to, derivatives, variants, and oligomers, as well as fusion proteins or fragments thereof.

SERPINE2 polypeptides include full length proteins encoded by the nucleic acid sequences set forth above. Particularly preferred SERPINE2 polypeptides comprise the amino acid sequence of SEQ ID NO:2.

The invention also provides polypeptides and fragments of the SERPINE2 that retain a desired biological activity, such as activation of collagen 1A1 or α-smooth muscle actin production or the generation of myofibroblasts from human lung fibroblasts. Such a fragment is preferably a soluble polypeptide.

Also provided herein are polypeptide fragments of varying lengths. In one embodiment, a preferred SERPINE2 polypeptide fragment comprises at least 6 contiguous amino acids of an amino acid sequence. In other embodiments, a preferred SERPINE2 polypeptide fragment comprises at least 10, at least 20, at least 30, up to at least 100 contiguous amino acids of the amino acid sequences of SEQ ID NO:2. These polypeptides can be produced in soluble form. Polypeptide fragments also can be employed as immunogens, in generating antibodies.

The invention encompasses variants of SERPINE2 and fragments thereof. Preferably, a variant of SERPINE2 comprises an amino acid sequence showing an identity of at least 50%, 70%, 75%, 80%, 85%, 90%, 95%, 97%, 98%, or 99% with SEQ ID NO:2 or a fragment thereof. Such a fragment can be, for example, of 50, 100, 150, 200, 250, 300, 350, or 375 amino acids in size.

The percent identity can be determined by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

### Production of Polypeptides and Fragments Thereof

Expression, isolation, and purification of the polypeptides and fragments of the invention can be accomplished by any suitable technique, including but not limited to the following.

Expression Systems

The present invention also provides recombinant cloning and expression vectors containing SERPINE2 DNA, as well as host cell containing the recombinant vectors. Expression vectors comprising SERPINE2 DNA can be used to prepare SERPINE2 polypeptides or fragments encoded by the DNA. A method for producing polypeptides comprises culturing host cells transformed with a recombinant expression vector encoding the polypeptide, under conditions that promote expression of the polypeptide, then recovering the expressed polypeptides from the culture. The skilled artisan will recognize that the procedure for purifying the expressed polypeptides will vary according to such factors as the type of host cells employed, and whether the polypeptide is membrane-bound or a soluble form that is secreted from the host cell.

Any suitable expression system can be employed. The vectors include a DNA encoding a SERPINE2 polypeptide or fragment of the invention, operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences that control transcription and translation initiation and termination. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) can be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide upon secretion of polypeptide from the cell.

Suitable host cells for expression of polypeptides include prokaryotes, yeast or higher eukaryotic cells. Mammalian or insect cells are generally preferred for use as host cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce polypeptides using RNAs derived from DNA constructs disclosed herein.

Prokaryotic Systems

Prokaryotes include gram-negative or gram-positive organisms. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a polypeptide can include an N-terminal methionine residue to facilitate expression of the recombinant polypeptide in the prokaryotic host cell. The N-terminal Met can be cleaved from the expressed recombinant polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. An appropriate promoter and a DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include betalactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776) and tac promoter (Maniatis, Molecular Cloning: A Laboratory Manual, Cold Spring Harbor Laboratory, p. 412, 1982). A particularly useful prokaryotic host cell expression system employs a phage lambdaPL promoter and a cl857ts thermolabile repressor sequence. Plasmid vectors available from the American Type Culture Collection which incorporate derivatives of the lambdaPL promoter include plasmid pHUB2 (resident in E. coli strain JMB9, ATCC 37092) and pPLc28 (resident in E. coli RR1, ATCC 53082).

SERPINE2 DNA can be cloned in-frame into the multiple cloning site of an ordinary bacterial expression vector. Ideally, the vector would contain an inducible promoter upstream of the cloning site, such that addition of an inducer leads to high-level production of the recombinant protein at a time of the investigator's choosing. For some proteins, expression levels can be boosted by incorporation of codons encoding a fusion partner (such as hexahistidine) between the promoter and the gene of interest. The resulting “expression plasmid” can be propagated in a variety of strains of E. coli.

For expression of the recombinant protein, the bacterial cells are propagated in growth medium until reaching a pre-determined optical density. Expression of the recombinant protein is then induced, e.g. by addition of IPTG (isopropyl-b-D-thiogalactopyranoside), which activates expression of proteins from plasmids containing a lac operator/promoter. After induction (typically for 1-4 hours), the cells are harvested by pelleting in a centrifuge, e.g. at 5,000×G for 20 minutes at 4° C.

For recovery of the expressed protein, the pelleted cells can be resuspended in ten volumes of 50 mM Tris-HCl (pH 8)/1 M NaCl and then passed two or three times through a French press. Most highly expressed recombinant proteins form insoluble aggregates known as inclusion bodies. Inclusion bodies can be purified away from the soluble proteins by pelleting in a centrifuge at 5,000×G for 20 minutes, 4° C. The inclusion body pellet is washed with 50 mM Tris-HCl (pH 8)/1% Triton X-100 and then dissolved in 50 mM Tris-HCl (pH 8)/8 M urea/0.1 M DTT. Any material that cannot be dissolved is removed by centrifugation (10,000×G for 20 minutes, 20° C.). The protein of interest will, in most cases, be the most abundant protein in the resulting clarified supernatant. This protein can be “refolded” into the active conformation by dialysis against 50 mM Tris-HCl (pH 8)/5 mM CaCl₂/5 mM Zn(OAc)₂/1 mM GSSG/0.1 mM GSH. After refolding, purification can be carried out by a variety of chromatographic methods, such as ion exchange or gel filtration. In some protocols, initial purification can be carried out before refolding. As an example, hexahistidine-tagged fusion proteins can be partially purified on immobilized Nickel.

While the preceding purification and refolding procedure assumes that the protein is best recovered from inclusion bodies, those skilled in the art of protein purification will appreciate that many recombinant proteins are best purified out of the soluble fraction of cell lysates. In these cases, refolding is often not required, and purification by standard chromatographic methods can be carried out directly.

Yeast Systems

Alternatively, the SERPINE2 polypeptides can be expressed in yeast host cells, preferably from the Saccharomyces genus (e.g., S. cerevisiae). Other genera of yeast, such as Pichia or Kluyveromyces, can also be employed. Yeast vectors will often contain an origin of replication sequence from a 2 μm yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase. Other suitable vectors and promoters for use in yeast expression are further described in Hitzeman, EPA-73,657. Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli can be constructed by inserting DNA sequences from pBR322 for selection and replication in E. coli (Amp^{r} gene and origin of replication) into the above-described yeast vectors.

The yeast alpha-factor leader sequence can be employed to direct secretion of the polypeptide. The alpha-factor leader sequence is often inserted between the promoter sequence and the structural gene sequence. See, e.g., Kurjan et al., Cell 30:933, 1982 and Bitter et al., Proc. Natl. Acad. Sci. USA 81:5330, 1984. Other leader sequences suitable for facilitating secretion of recombinant polypeptides from yeast hosts are known to those of skill in the art. A leader sequence can be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 mg/ml adenine and 20 mg/ml uracil.

Yeast host cells transformed by vectors containing an ADH2 promoter sequence can be grown for inducing expression in a “rich” medium. An example of a rich medium is one consisting of 1% yeast extract, 2% peptone, and 1% glucose supplemented with 80 mg/ml adenine and 80 mg/ml uracil. Derepression of the ADH2 promoter occurs when glucose is exhausted from the medium.

Mammalian or Insect Systems

Mammalian or insect host cell culture systems also can be employed to express recombinant SERPINE2 polypeptides. Bacculovirus systems for production of heterologous proteins in insect cells are reviewed by Luckow and Summers, Bio/Technology 6:47 (1988). Established cell lines of mammalian origin also can be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991).

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Feigner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable host strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 by sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534 and PCT Application WO 97/25420) and the tripartite leader (TPL) and VA gene RNAs from Adenovirus 2 (Gingeras et al., J. Biol. Chem. 257:13475-13491, 1982). The internal ribosome entry site (IRES) sequences of viral origin allows dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (e.g. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., Biotechniques 22:150-161, 1997, and p2A5I described by Morris et al., Animal Cell Technology, 1997, pp. 529-534.

Other expression vectors for use in mammalian host cells can be constructed as disclosed by Okayama and Berg (Mol. Cell. Biol. 3:280, 1983). In yet another alternative, the vectors can be derived from retroviruses. An additional useful expression vector is p FLAG®. FLAG® technology is centered on the fusion of a low molecular weight (1 kD), hydrophilic, FLAG® marker peptide to the N-terminus of a recombinant protein expressed by pFLAG® expression vectors.

Purification

The invention also includes methods of isolating and purifying the polypeptides and fragments thereof. An isolated and purified SERPINE2 polypeptide according to the invention can be produced by recombinant expression systems as described above or purified from naturally occurring cells. SERPINE2 polypeptide can be substantially purified, as indicated by a single protein band upon analysis by SDS-polyacrylamide gel electrophoresis (SDS-PAGE). One process for producing SERPINE2 comprises culturing a host cell transformed with an expression vector comprising a DNA sequence that encodes a SERPINE2 polypeptide under conditions sufficient to promote expression of SERPINE2. The SERPINE2 polypeptide is then recovered from culture medium or cell extracts, depending upon the expression system employed.

Exemplary methods for the purification of SERPINE2 polypeptides are known in the art. For example, SERPINE2 polypeptides can be isolated and purified by hollow fiber filtration followed by recirculation on a heparin-sepharose column. Howard et al., J. Biol. Chem. 261:684-689, 1986; Scott et al., J. Biol. Chem. 258:10439-10444, 1983; Scott et al., J. Biol. Chem. 258:4397-4403, 1983. Affinity chromatography using specific polyclonal antibodies against SERPINE2 (Howard et al., 1986) can also be employed.

Isolation and Purification

The expression “isolated and purified” as used herein means that SERPINE2 is essentially free of association with other host DNA, proteins, or polypeptides, for example, as a purification product of recombinant host cell culture or as a purified product from a non-recombinant source. An “isolated and purified” SERPINE2 protein can include other proteins added to the SERPINE2 to stabilize or assist with purification of the SERPINE2 of the protein, such as albumin. The term “substantially purified” as used herein refers to a mixture that contains SERPINE2 and is essentially free of association with other DNA, proteins, or polypeptides, but for the presence of known DNA or proteins that can be removed using a specific antibody, and which substantially purified SERPINE2 proteins retain biological activity. The term “purified SERPINE2” refers to either the “isolated and purified” form of SERPINE2 or the “substantially purified” form of SERPINE2, as both are described herein.

The term “biologically active” as it refers to SERPINE2 protein, means that the SERPINE2 protein is capable of associating with SERPINE2 target trypsin-like serine proteases, such as thrombin, trypsin, plasmin, and urokinase, and inactivating them.

In one preferred embodiment, the purification of recombinant polypeptides or fragments can be accomplished using fusions of SERPINE2 polypeptides or fragments to another polypeptide to aid in the purification of polypeptides or fragments. Such fusion partners can include poly-His, Fc moieties, or other antigenic identification peptides.

With respect to any type of host cell, as is known to the skilled artisan, procedures for purifying a recombinant polypeptide or fragment will vary according to such factors as the type of host cells employed and whether or not the recombinant polypeptide or fragment is secreted into the culture medium.

In general, the recombinant SERPINE2 polypeptide or fragment can be isolated from the host cells if not secreted, or from the medium or supernatant if soluble and secreted, followed by one or more concentration, salting-out, ion exchange, hydrophobic interaction, affinity purification, or size exclusion chromatography steps. As to specific ways to accomplish these steps, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. In addition, a chromatofocusing step can be employed. Alternatively, a hydrophobic interaction chromatography step can be employed. Suitable matrices can be phenyl or octyl moieties bound to resins. In addition, affinity chromatography with a matrix which selectively binds the recombinant protein can be employed. Examples of such resins employed are heparin columns, lectin columns, dye columns, and metal-chelating columns. Finally, one or more reversed-phase high performance liquid chromatography (RP-HPLC) steps employing hydrophobic RP-HPLC media, (e.g., silica gel or polymer resin having pendant methyl, octyl, octyldecyl or other aliphatic groups) can be employed to further purify the polypeptides. Some or all of the foregoing purification steps, in various combinations, are well known and can be employed to provide an isolated and purified recombinant protein.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

It is also possible to utilize an affinity column comprising a SERPINE2 binding protein, such as a monoclonal antibody generated against SERPINE2 polypeptides, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention.

The desired degree of purity depends on the intended use of the protein. A relatively high degree of purity is desired when the SERPINE2 polypeptide is to be administered in vivo, for example. In such a case, the SERPINE2 polypeptides are purified such that no protein bands corresponding to other proteins are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single protein band upon analysis by SDS-PAGE. The protein band can be visualized by silver staining, Coomassie blue staining, or (if the protein is radiolabeled) by autoradiography.

Purified preparations of SERPINE2 are commercially available and can be used in the methods of the invention.

### Antagonists of SERPINE2

The invention encompasses antagonists of SERPINE2. An antagonist of SERPINE2 interferes with SERPINE2 function, for example, by abrogating the protease inhibitory function of SERPINE2. In preferred embodiments, the antagonist downregulates, or decreases, collagen 1A1 expression caused by elevated levels of SERPINE2. In preferred embodiments, the antagonist downregulates, or decreases, α-smooth muscle actin expression caused by elevated levels of SERPINE2. Most preferably, the antagonist downregulates, or decreases, collagen 1A1 and α-smooth muscle actin expression caused by elevated levels of SERPINE2. Preferably, the downregulation is in lung fibroblasts, most preferably human lung fibroblasts. Expression can be measured directly, by measuring RNA, or indirectly, for example, by measuring protein.

Such antagonists include antibodies which specifically bind to SERPINE2 and inhibit SERPINE2 biological activity; antisense nucleic acids RNAs that interfere with the expression of SERPINE2; small interfering RNAs that interfere with the expression of SERPINE2; small peptides corresponding to the reactive center loop of SERPINE2; and small molecule inhibitors of SERPINE2.

Candidate antagonists of SERPINE2 can be screened for function by a variety of techniques known in the art and/or disclosed within the instant application, such as ability to interfere with inhibition by SERPINE2 of trypsin-like serine proteases, such as thrombin, trypsin, plasmin, and urokinase; inhibition of collagen and/or α-smooth muscle actin expression in vitro; and protection against bleomycin-induced fibrosis in a mouse model.

### Antibodies

In one embodiment, the antagonist of SERPINE2 is an antibody. Antibodies can be synthetic, monoclonal, or polyclonal and can be made by techniques well known in the art. Such antibodies specifically bind to SERPINE2 via the antigen-binding sites of the antibody (as opposed to non-specific binding). The SERPINE2 polypeptides, fragments, variants, fusion proteins, etc., as set forth above can be employed as immunogens in producing antibodies immunoreactive therewith. More specifically, the polypeptides, fragment, variants, fusion proteins, etc. contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hinderances, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes can be identified by any of the methods known in the art.

Thus, one aspect of the present invention relates to the antigenic epitopes of SERPINE2 polypeptides. Such epitopes are useful for raising antibodies, in particular monoclonal antibodies, as described in detail below. Additionally, epitopes from SERPINE2 polypeptides can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

Antibodies, including scFV fragments, that bind specifically to SERPINE2 and block its inhibition of target proteases are encompassed by the invention. Such antibodies can be generated, for example, using the procedures set forth in Verbeke et al., J. Thromb. Haemost. 2:298-305, 2004 and Brooks et al., Clinical & Experimental Metastasis 18:445-453, 2001.

The invention encompasses monoclonal antibodies against SERPINE2 that block its inhibition of target proteases. Exemplary blocking monoclonal antibodies against SERPINE2 are described in Wagner et al., Biochemistry 27: 2173-2176, 1988, and Boulaftali et al. Blood First Edition Paper, prepublished online Oct. 23, 2009; DOI 10.1182/blood-2009-04-217240.

In particular, monoclonal antibodies that block the binding of SERPINE2 to its target proteases or block the binding of SERPINE2 to heparin are preferred. Such antibodies can be screened using routine in vitro binding assays or using the assays set forth in the examples.

In one embodiment a monoclonal antibody is generated that binds to the protease interaction domain of SERPINE2. This antibody can be generated using a complete SERPINE2 polypeptide or a fragment of SERPINE2 containing the protease interaction domain of SERPINE2 as an immunogen. The antibody can be assessed for its ability to block the interaction of SERPINE2 with a target protease.

Antibodies are capable of binding to their targets with both high avidity and specificity. They are relatively large molecules (−150 kDa), which can sterically inhibit interactions between two proteins (e.g. SERPINE2 and its target protease) when the antibody binding site falls within proximity of the protein-protein interaction site. Thus, in one embodiment, the invention encompasses an antibody which binds to the “reactive centre loop” (RCL) of SERPINE2 and inhibits binding of the cognate protease can prevent its inactivation by SERPINE2. The invention encompasses antibodies that bind to RCL residues that directly contact the protease. The invention further encompasses antibodies that bind to epitopes within close proximity to the SERPINE2-protease binding site.

In various embodiments, the invention encompasses antibodies which bind residues that contact SERPINE2 co-factors, such as heparin, or to residues in the proximity of co-factor binding sites that impair SERPINE2 inhibitory activity by interfering with co-factor mediated enhancement of SERPINE2 inhibitory activity.

In various embodiments, the invention encompasses antibodies that interfere with intermolecular interactions (e.g. protein-protein interactions), as well as antibodies that perturb intramolecular interactions (e.g. conformational changes within a molecule). Thus, antibodies which binds to the RCL of SERPINE2, preferably to amino acids 348 to 364 or amino acids 348 to 374 of SERPINE2, and prevent insertion of the loop into “beta-sheet A” following protease binding and prevent SERPINE2 inhibitory activity by interfering with the distortion and subsequent degradation of the attached protease are encompassed by the invention. Similarly, antibodies that compel the RCL of unoccupied SERPINE2 to adapt an “inserted” conformation and interfere with serpin activity by keeping protease binding sites sequestered and unavailable for protease binding are encompassed by the invention.

The ability of antibodies to bind specific targets has been exploited to deliver specifically various types of functional molecules to a target of interest. Examples of such molecules include toxins, cytokines, radioisotopes, and small-molecule drugs or pro-drugs. In such cases, these molecules may be attached to the antibody via covalent chemical or peptide linkers, or in the case of polypeptides such as cytokines, they may be directly attached via a peptide bond. Similarly, antibodies targeting SERPINE2 can be used to deliver molecules that specifically inactivate its protease inhibitor activity. In one embodiment, the invention encompasses an antibody which delivers a mutated protease directly to SERPINE2. This mutated protease can retain protease activity, form the covalent ester bond with SERPINE2, cleave the RCL, and induce RCL insertion into the beta sheet, but does not retain the ability to bind (and thus cleave) its own native substrate. Since SERPINE2 binds its cognate protease with a 1:1 molar ratio, and since the SERPINE2 is itself destroyed when it binds to and inactivates the protease, delivery of this mutated protease to SERPINE2 by an antibody can effectively exhaust the supply of SERPINE2, increasing the level of native cognate protease activity. Mutated protease can be attached to the antibody via co-translational or post-translational means.

Antibodies can be screened for the ability to block the biological activity of SERPINE2, or the binding of SERPINE2 to a ligand, and/or for other properties. For example, antibodies can be screened for the ability to bind and block trypsin-like serine proteases, such as thrombin, trypsin, plasmin, and urokinase in vitro. See, e.g., Wagner et al., Biochemistry 27: 2173-2176, 1988. Also, antibodies can be screened for the ability to block myofibroblast formation or to inhibit collagen 1A1 and/or α-smooth muscle actin expression in human lung fibroblast cells exposed to elevated levels of SERPINE2 using the procedures set forth herein. Further, antibodies can be screened for the ability to protect in vivo against bleomycin-induced pulmonary fibrosis using the mouse model described in Yaekashiwa et al., Am. J. Respir. Crit. Care Med. 156:1937-1944 (1997) and Dohi et al., Am. J. Respir. Crit. Care Med. 162:2302-2307 (2000).

As to the antibodies that can be elicited by the epitopes of SERPINE2 polypeptides, whether the epitopes have been isolated or remain part of the polypeptides, both polyclonal and monoclonal antibodies can be prepared by conventional techniques as described below.

In this aspect of the invention, SERPINE2 and peptides based on the amino acid sequence of SERPINE2, can be utilized to prepare antibodies that specifically bind to SERPINE2. The term “antibodies” is meant to include polyclonal antibodies, monoclonal antibodies, fragments thereof, such as F(ab′)₂ and Fab fragments, single-chain variable fragments (scFvs), single-domain antibody fragments (VHHs or Nanobodies), bivalent antibody fragments (diabodies), as well as any recombinantly and synthetically produced binding partners.

Antibodies are defined to be specifically binding if they bind SERPINE2 polypeptide with a Ka of greater than or equal to about 10⁷ M⁻¹. Affinities of binding partners or antibodies can be readily determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice, or rats, using procedures that are well known in the art. In general, purified SERPINE2 or a peptide based on the amino acid sequence of SERPINE2 that is appropriately conjugated is administered to the host animal typically through parenteral injection. The immunogenicity of SERPINE2 can be enhanced through the use of an adjuvant, for example, Freund's complete or incomplete adjuvant. Following booster immunizations, small samples of serum are collected and tested for reactivity to SERPINE2 polypeptide. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980.

For example, the host animals, such as mice, can be injected intraperitoneally at least once and preferably at least twice at about 3 week intervals with isolated and purified SERPINE2 or conjugated SERPINE2 peptide, for example a peptide comprising or consisting of amino acids 348 to 364 or amino acids 348 to 374, optionally in the presence of adjuvant. Mouse sera are then assayed by conventional dot blot technique or antibody capture (ABC) to determine which animal is best to fuse. Approximately two to three weeks later, the mice are given an intravenous boost of SERPINE2 or conjugated SERPINE2 peptide. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as a labeled SERPINE2 polypeptide, is added to each well followed by incubation. Positive wells can be subsequently detected. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Antigen-binding fragments of such antibodies, which can be produced by conventional techniques, are also encompassed by the present invention. Examples of such fragments include, but are not limited to, Fab and F(ab′)₂ fragments. Antibody fragments and derivatives produced by genetic engineering techniques are also provided.

The monoclonal antibodies of the present invention include chimeric antibodies, e.g., humanized versions of murine monoclonal antibodies. Such humanized antibodies can be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993). Procedures to generate antibodies transgenically can be found in GB 2,272,440, U.S. Pat. Nos. 5,569,825 and 5,545,806.

Antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Publication No. WO 87/02671; Akira, et al. European Patent Application 0184187; Taniguchi, M., European Patent Application 0171496; Morrison et al. European Patent Application 0173494; Neuberger et al. PCT International Publication No. WO 86/01533; Cabilly et al. U.S. Pat. No. 4,816,567; Cabilly et al. European Patent Application 0125023; Better et al., Science 240:1041 1043, 1988; Liu et al., PNAS 84:3439 3443, 1987; Liu et al., J. Immunol. 139:3521 3526, 1987; Sun et al. PNAS 84:214 218, 1987; Nishimura et al., Canc. Res. 47:999 1005, 1987; Wood et al., Nature 314:446 449, 1985; and Shaw et al., J. Natl. Cancer Inst. 80:1553 1559, 1988); Morrison, S. L., Science 229:1202 1207, 1985; Oi et al., BioTechniques 4:214, 1986; Winter U.S. Pat. No. 5,225,539; Jones et al., Nature 321:552 525, 1986; Verhoeyan et al., Science 239:1534, 1988; and Beidler et al., J. Immunol. 141:4053 4060, 1988.

In connection with synthetic and semi-synthetic antibodies, such terms are intended to cover but are not limited to antibody fragments, isotype switched antibodies, humanized antibodies (e.g., mouse-human, human-mouse), hybrids, antibodies having plural specificities, and fully synthetic antibody-like molecules.

In a preferred embodiment, the antagonist is an antibody which specifically recognizes the active site (i.e., the reactive center loop) of SERPINE2. For therapeutic applications, “human” monoclonal antibodies having human constant and variable regions are often preferred so as to minimize the immune response of a patient against the antibody. Such antibodies can be generated by immunizing transgenic animals which contain human immunoglobulin genes. See Jakobovits et al. Ann NY Acad Sci 764:525-535 (1995).

Human monoclonal antibodies against SERPINE2 polypeptides can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581 597; and Griffths et al. (1993) EMBO J. 12:725 734. In addition, a combinatorial library of antibody variable regions can be generated by mutating a known human antibody. For example, a variable region of a human antibody known to bind SERPINE2, can be mutated, by for example using randomly altered mutagenized oligonucleotides, to generate a library of mutated variable regions which can then be screened to bind to SERPINE2. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457 4461.

An immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370 1372; Hay et al. (1992) Hum Antibod Hybridomas 3:81 85; Huse et al. (1989) Science 246:1275 1281; Griffths et al. (1993) supra; Hawkins et al. (1992) J Mol Biol 226:889 896; Clackson et al. (1991) Nature 352:624 628; Gram et al. (1992) PNAS 89:3576 3580; Garrad et al. (1991) Bio/Technology 9:1373 1377; Hoogenboom et al. (1991) Nuc Acid Res 19:4133 4137; and Barbas et al. (1991) PNAS 88:7978 7982. Once displayed on the surface of a display package (e.g., filamentous phage), the antibody library is screened to identify and isolate packages that express an antibody that binds a SERPINE2 polypeptide. In a preferred embodiment, the primary screening of the library involves panning with an immobilized SERPINE2 polypeptide and display packages expressing antibodies that bind immobilized SERPINE2 polypeptide are selected.

### Organic and Peptide Small Molecule Inhibitors

In other embodiments of the invention, antagonists are used which are peptides, polypeptides, proteins, or peptidomimetics designed as ligands for SERPINE2 on the basis of the presence of their ability to bind to the active site (i.e., the reactive center loop) of SERPINE2. The design of such molecules as ligands for the integrins is exemplified, for example, in Pierschbacher et al., J. Cell. Biochem. 56:150-154 (1994)); Chorev et al. Biopolymers 37:367-375 (1995)); Pasqualini et al., J. Cell. Biol. 130:1189-1196 (1995)); and Smith et al., J. Biol, Chem, 269:32788-32795 (1994)).

Exemplary procedures for the inactivation of SERPINE2 using an amino acid peptide corresponding to the reactive center loop are provided in Eitzman et al., J. Cin. Invest. 95:2416-2420, 1995; Bjork et al., J. Biol. Chem. 267:1976-1982, 1992; and Schulze et al., Eur. J. Biochem. 194:51-56, 1990.

In other embodiments of the invention, antagonists are used which are low molecular weight organic molecules that inactivate or inhibit SERPINE2 activity. Exemplary procedures for the use of low molecular weight organic molecules for the inactivation of SERPINE2 are provided in Brooks et al., Anticancer Drugs 15:37-44, 2004, and in U.S. Pat. Nos. 7,368,471, 7,259,182, and 6,599,925, which provide low molecular weight organic molecules for the inactivation of SERPINE1.

### Antisense Nucleic Acid Molecules

In some embodiments of the invention, antisense nucleic acid molecules are used as antagonists of SERPINE2. Antisense nucleic acid molecules are complementary oligonucleotide strands of nucleic acids designed to bind to a specific sequence of nucleotides to inhibit production of a targeted protein.

Antisense or sense oligonucleotides, according to the present invention, comprise a fragment of DNA (SEQ ID NO:1). Such a fragment generally comprises at least about 14 nucleotides, preferably from about 14 to about 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Antisense RNAs and oligonucleotides can be made and employed to inhibit SERPINE2 expression as described in Kim and Loh, Mol. Biol. Cell. 17:789-798, 2006, and Sawa et al., J. Biol. Chem. 269:14149-14152, 1994.

Given the coding strand sequences encoding these components, antisense nucleic acids can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Binding of antisense or sense oligonucleotides to target nucleic acid sequences results in the formation of duplexes that block or inhibit protein expression by one of several means, including enhanced degradation of the mRNA by RNAseH, inhibition of splicing, premature termination of transcription or translation, or by other means. The antisense oligonucleotides thus can be used to block expression of SERPINE2. Antisense or sense oligonucleotides further comprise oligonucleotides having modified sugar-phosphodiester backbones (or other sugar linkages, such as those described in WO91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L-lysine). Further still, intercalating agents, such as ellipticine, and alkylating agents or metal complexes can be attached to sense or antisense oligonucleotides to modify binding specificities of the antisense or sense oligonucleotide for the target nucleotide sequence.

Antisense or sense oligonucleotides can be introduced into a cell containing the target nucleic acid sequence by any gene transfer method, including, for example, lipofection, CaPO₄-mediated DNA transfection, electroporation, or by using gene transfer vectors such as Epstein-Barr virus.

Sense or antisense oligonucleotides are preferably introduced into a cell containing the target nucleic acid sequence by insertion of the sense or antisense oligonucleotide into a suitable retroviral vector, then contacting the cell with the retrovirus vector containing the inserted sequence, either in vivo or ex vivo. Suitable retroviral vectors include the murine retrovirus M-MuLV, N2 (a retrovirus derived from M-MuLV), or the double copy vectors designated DCT5A, DCT5B and DCT5C (see PCT Application U.S. Ser. No. 90/02656).

Sense or antisense oligonucleotides also can be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide can be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

The antisense antagonist can be provided as an antisense oligonucleotide such as RNA (see, for example, Murayama et al. Antisense Nucleic Acid Drug Dev. 7:109-114 (1997)). Antisense genes can also be provided in a viral vector, such as, for example, in hepatitis B virus (see, for example, Ji et al., J. Viral Hepat. 4:167-173 (1997)); in adeno-associated virus (see, for example, Xiao et al. Brain Res. 756:76-83 (1997)); or in other systems including but not limited to an HVJ (Sendai virus)-liposome gene delivery system (see, for example, Kaneda et al. Ann, N.Y. Acad. Sci. 811:299-308 (1997)); a “peptide vector” (see, for example, Vidal et al. CR Acad. Sci. III 32):279-287 (1997)); as a gene in an episomal or plasmid vector (see, for example, Cooper et al. Proc. Natl. Acad. Sci. U.S.A. 94:6450-6455 (1997), Yew et al. Hum Gene Ther 8:575-584 (1997)); as a gene in a peptide-DNA aggregate (see, for example, Niidome et al. J. Biol. Chem. 272:15307-15312 (1997)); as “naked DNA” (see, for example, U.S. Pat. Nos. 5,580,859 and 5,589,466); and in lipidic vector systems (see, for example, Lee et al. Crit. Rev Ther Drug Carrier Syst, 14:173-206 (1997))

### Small Interfering RNAs

In some embodiments of the invention, RNAi molecules are used as antagonists of SERPINE2. RNAi regulates gene expression via a ubiquitous mechanism by degradation of target mRNA in a sequence-specific manner. McManus et al., 2002, Nat Rev Genet 3:737 747. In mammalian cells, interfering RNA (RNAi) can be triggered by 21- to 23-nucleotide duplexes of siRNA. Lee et al., 2002, Nat Biotechnol 20: 500 505; Paul et al., 2002, Nat. Biotechnol. 20:505 508; Miyagishi et al., 2002, Nat. Biotechnol. 20:497 500; Paddison et al., 2002, Genes Dev. 16: 948 958. The expression of siRNA or short hairpin RNA (shRNA) driven by U6 promoter effectively mediates target mRNA degradation in mammalian cells. Synthetic siRNA duplexes and plasmid-derived siRNAs can inhibit HIV-1 infection and replication by specifically degrading HIV genomic RNA. McManus et al., J. Immunol. 169:5754 5760; Jacque et al., 2002, Nature 418:435 438; Novina et al., 2002, Nat Med 8:681 686. Also, siRNA targeting HCV genomic RNA inhibits HCV replication. Randall et al., 2003, Proc Natl Acad Sci USA 100:235 240; Wilson et al., 2003, Proc Natl Acad Sci USA 100: 2783 2788. Fas targeted by siRNA protects the liver from fulminant hepatitis and fibrosis. Song et al., 2003, Nat Med 9:347 351.

In preferred embodiments, an RNA interference (RNAi) molecule is used to decrease gene expression of SERPINE2. RNA interference (RNAi) refers to the use of double-stranded RNA (dsRNA) or small interfering RNA (siRNA) to suppress the expression of a gene comprising a related nucleotide sequence. RNAi is also called post-transcriptional gene silencing (or PTGS). Since the only RNA molecules normally found in the cytoplasm of a cell are molecules of single-stranded mRNA, the cell has enzymes that recognize and cut dsRNA into fragments containing 21-25 base pairs (approximately two turns of a double helix and which are referred to as small interfering RNA or siRNA). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Exemplary procedures for the use of RNA interference to suppress SERPINE2 expression is provided Kortlever et al., Nature Cell Biology 8:877-884, 2006.

Double-stranded (ds) RNA can be used to interfere with gene expression in mammals. dsRNA is used as inhibitory RNA or RNAi of the function of a nucleic acid molecule of the invention to produce a phenotype that is the same as that of a null mutant of a SERPINE2 nucleic acid molecule (see Wianny & Zernicka-Goetz, 2000, Nature Cell Biology 2: 70 75).

Alternatively, siRNA can be introduced directly into a cell to mediate RNA interference (Elbashir et al., 2001, Nature 411:494 498). Many methods have been developed to make siRNA, e.g, chemical synthesis or in vitro transcription. Once made, the siRNAs are introduced into cells via transient transfection. A number of expression vectors have also been developed to continually express siRNAs in transiently and stably transfected mammalian cells (Brummelkamp et al., 2002 Science 296:550 553; Sui et al., 2002, PNAS 99(6):5515 5520; Paul et al., 2002, Nature Biotechnol. 20:505 508). Some of these vectors have been engineered to express small hairpin RNAs (shRNAs), which get processed in vivo into siRNA-like molecules capable of carrying out gene-specific silencing. Another type of siRNA expression vector encodes the sense and antisense siRNA strands under control of separate pol 111 promoters (Miyagishi and Taira, 2002, Nature Biotechnol. 20:497 500). The siRNA strands from this vector, like the shRNAs of the other vectors, have 3′ thymidine termination signals. Silencing efficacy by both types of expression vectors was comparable to that induced by transiently transfecting siRNA.

RNA can be directly introduced into the cell (i.e., intracellularly); or introduced extracellularly into a cavity, interstitial space, into the circulation of an organism, or introduced orally. Physical methods of introducing nucleic acids, for example, injection directly into the cell or extracellular injection into the organism, can also be used. Vascular or extravascular circulation, the blood or lymph system, and the cerebrospinal fluid are sites where the RNA can be introduced.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells can be used, such as lipid-mediated carrier transport, chemical-mediated transport, such as calcium phosphate, and the like. Thus, the RNA can be introduced along with components that perform one or more of the following activities: enhance RNA uptake by the cell, promote annealing of the duplex strands, stabilize the annealed strands, or otherwise increase inhibition of the target gene.

The RNA can comprise one or more strands of polymerized ribonucleotide. It can include modifications to either the phosphate-sugar backbone or the nucleoside. For example, the phosphodiester linkages of natural RNA can be modified to include at least one of a nitrogen or sulfur heteroatom. Modifications in RNA structure can be tailored to allow specific genetic inhibition while avoiding a general panic response in some organisms which is generated by dsRNA. Likewise, bases can be modified to block the activity of adenosine deaminase. RNA can be produced enzymatically or by partial/total organic synthesis, any modified ribonucleotide can be introduced by in vitro enzymatic or organic synthesis.

The double-stranded structure can be formed by a single self-complementary RNA strand or two complementary RNA strands. RNA duplex formation can be initiated either inside or outside the cell. The RNA can be introduced in an amount which allows delivery of at least one copy per cell. Higher doses (e.g., at least 5, 10, 100, 500 or 1000 copies per cell) of double-stranded material can yield more effective inhibition; lower doses can also be useful for specific applications. Inhibition is sequence-specific in that nucleotide sequences corresponding to the duplex region of the RNA are targeted for genetic inhibition. The RNA molecule can be at least 10, 12, 15, 20, 21, 22, 23, 24, 25, 30, nucleotides in length.

RNA containing a nucleotide sequences identical to a portion of the target gene are preferred for inhibition. RNA sequences with insertions, deletions, and single point mutations relative to the target sequence have also been found to be effective for inhibition. Thus, sequence identity can be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA can be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences can be at least 25, 50, 100, 200, 300 or 400 bases.

One hundred percent sequence identity between the RNA and the target gene is not required to practice the present invention. Thus the invention has the advantage of being able to tolerate sequence variations that might be expected due to genetic mutation, strain polymorphism, or evolutionary divergence.

RNA can be synthesized either in vivo or in vitro. Endogenous RNA polymerase of the cell can mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) can be used to transcribe the RNA strand (or strands). Inhibition can be targeted by specific transcription in an organ, tissue, or cell type; stimulation of an environmental condition (e.g., infection, stress, temperature, chemical inducers); and/or engineering transcription at a developmental stage or age. The RNA strands can be polyadenylated; the RNA strands can be capable of being translated into a polypeptide by a cell's translational apparatus. RNA can be chemically or enzymatically synthesized by manual or automated reactions. The RNA can be synthesized by a cellular RNA polymerase or a bacteriophage RNA polymerase (e.g., T3, T7, SP6). The use and production of an expression construct are known in the art (see also WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA can be purified prior to introduction into the cell. For example, RNA can be purified from a mixture by extraction with a solvent or resin, precipitation, electrophoresis, chromatography, or a combination thereof. Alternatively, the RNA can be used with no or a minimum of purification to avoid losses due to sample processing. The RNA can be dried for storage or dissolved in an aqueous solution. The solution can contain buffers or salts to promote annealing, and/or stabilization of the duplex strands.

The present invention can be used to introduce RNA into a cell for the treatment or prevention of disease, such as IPF. For example, dsRNA can be introduced into a human lung fibroblast cell and thereby inhibit gene expression of SERPINE2. Treatment would include amelioration of any symptom associated with the disease or clinical indication associated with the pathology.

### Formulation and Administration

A multitude of appropriate formulations of SERPINE2 antagonists can be found in the formulary known to all pharmaceutical chemists: Remington's Pharmaceutical Sciences, (15th Edition, Mack Publishing Company, Easton, Pa., (1975)), particularly Chapter 87, by Blaug, Seymour, therein. These formulations include for example, powders, pastes, ointments, jelly, waxes, oils, lipids, anhydrous absorption bases, oil-in-water or water-in-oil emulsions, emulsions carbowax (polyethylene glycols of a variety of molecular weights), semi-solid gels, and semi-solid mixtures containing carbowax.

The invention includes the use of an antagonist of SERPINE2 for the preparation of a medicament for the treatment of a medical condition, particularly, lung fibrosis, especially one in which human lung fibroblast cells are exposed to an elevated level of SERPINE2. In preferred embodiments, the medical condition is ALI, IPF, COPD, asthma, or ARDS. Preferably, the antagonist of SERPINE2 is an antibody, e.g., a monoclonal antibody, an RNAi molecule, an antisense nucleic acid molecule, a peptide, or a small molecule inhibitor of SERPINE2.

The invention includes methods of treating a patient with lung fibrosis comprising administering an antagonist of SERPINE2 to the patient. Preferably, the lung fibrosis is one in which human lung fibroblast cells are exposed to an elevated level of SERPINE2. In preferred embodiments, the patient has ALI, IPF, COPD, asthma, or ARDS. Preferably, the antagonist of SERPINE2 is an antibody, e.g., a monoclonal antibody, an RNAi molecule, an antisense nucleic acid molecule, a peptide, or a small molecule inhibitor of SERPINE2.

In various embodiments, an effective dose of the compositions of the invention is administered to the subject once a month or more than once a month, for example, every 2, 3, 4, 5, or 6 months. In other embodiments, an effective dose of the compositions of the invention is administered less than once a month, such as, for example, every two weeks or every week. An effective dose of the compositions of the invention is administered to the subject at least once. In certain embodiments, the effective dose of the composition may be administered multiple times, including for periods of at least a month, at least six months, or at least a year.

In various embodiments, the compositions of the invention are administered on a daily basis for at least a period of 1-5 days, although patients with established pulmonary fibrosis can receive therapeutic doses for periods of months to years. As used herein, “therapeutic dose” is a dose which prevents, alleviates, abates, or otherwise reduces the severity of symptoms in a patient.

Since SERPINE2 is an extracellular protease inhibitor, the extracellular administration of an antagonistic protein (e.g., antibody or peptide) or small molecule is sufficient to inhibit SERPINE2 function. The inhibition of SERPINE2 expression (e.g., antisense or RNAi) requires that the antagonist enters a cell in which SERPINE2 is expressed. In a preferred embodiment, the cell is a human lung fibroblast.

Various modes of delivery of medicaments to IPF patients are known in the art. For example, numerous clinical studies have been performed using various exemplary modes of delivery of molecules to treat IPF. Single IV infusion of an anti-connective tissue growth factor-specific monoclonal antibody has been used to treat IPF in a clinical study. Additionally, inhalation of small-molecules and subcutaneous injection and aerosol inhalation of Interferon-gamma have been employed in clinical studies. Furthermore, etanercept has been used to treat IPF by subcutaneous injection twice weekly in a clinical study. Raghu et al., Am J Respir Crit. Care Med. 178:948-55, 2008.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

The quantities of active ingredient necessary for effective therapy will depend on many different factors, including means of administration, target site, physiological state of the patient, and other medicaments administered. Thus, treatment dosages should be titrated to optimize safety and efficacy. Typically, dosages used in vitro can provide useful guidance in the amounts useful for in situ administration of the active ingredients. Animal testing of effective doses for treatment of particular disorders will provide further predictive indication of human dosage. Various considerations are described, for example, in Goodman and Gilman's the Pharmacological Basis of Therapeutics, 7th Edition (1985), MacMillan Publishing Company, New York, and Remington's Pharmaceutical Sciences 18th Edition, (1990) Mack Publishing Co, Easton Pa. Methods for administration are discussed therein, including oral, intravenous, intraperitoneal, intramuscular, transdermal, nasal, iontophoretic administration, and the like. Preferably, the formulation is administered into the lung. More preferably, the formulation is inhaled.

Preferably, local delivery to the lung is employed to alleviate potential side effects that can occur with systemic delivery. In this way, the dose that can be delivered locally can be substantially higher than what might be tolerated in a systemic (e.g. parental) delivery mode. For lung diseases such as idiopathic pulmonary fibrosis, cystic fibrosis, tuberculosis, pulmonary tumors or other inflammation, local delivery via the inhalation route is preferred. Intravenous administration is also preferred.

Delivery of small molecules to the lungs can be accomplished by techniques known in the art. In addition, protein drugs can be delivered to the lungs via inhalation by techniques known in the art. For example, protein drugs that have been delivered locally to the lungs exhibit a range of molecular weights, from insulin to antibodies.

While insulin is the best known example of an inhalable protein (Exubera), there are many examples of proteins targeted to the lungs where systemic delivery is undesirable. One of the oldest examples is interferon alpha or gamma which has been aerosolized to treat pulmonary tuberculosis (Am J Respir Crit. Care Med Vol 158. pp 1156-1162, 1998; Antimicrobial Agents and Chemotherapy, June 1984, p. 729-734). Today, aerosol interferon gamma is currently in Phase 1 clinical trials for idiopathic pulmonary fibrosis. Subcutaneous delivery was shown to be ineffective for this indication. Aerosol droplets of interferon are generally in the range of 0.3-3.4 uM using jet nebulizers with compressed air. The small particle size ensures exposure deep into the lung.

Larger proteins such as antibodies can also be delivered directly to the lung. For example, aerosolized monoclonal antibodies specific for T-cell receptors have been used successfully in pre-clinical studies for airway inflammation and hyperreactivity. (Intl Archives of Allergy and Immunology, 134, 49-55, 2004). In another example, aerosolized antibody against ricin toxin was found to protect the lungs of animals that inhaled the toxin (Toxicon, 34, 1037-1033, 1996). The animals receiving a control antibody developed airway epithelial necrosis with severe edema and inflammation of all lung lobes and died 48-96 hours post-ricin. In contrast, the animals given the aerosolized anti-ricin antibody did not develop lung lesions, and all the animals survived.

There are numerous devices that can be used to aid lung delivery such as nebulizers and atomizers for liquid formulations. Dry powder inhalers can be used for solid particle formulations. The existing devices can deliver in “active” or “passive” mode.

In one embodiment, antibodies against SERPINE2 can be directly nebulized from liquid solution as one route of delivery to the lung. In another embodiment, the antibodies against SERPINE2 can be mixed or encapsulated with a solid particle such as liposomes or poly-lactide microspheres (GRAS materials). The porous particles enable very high drug loads and can also provide for slow sustained release of the drug. The particles can be made in uniform size, with 5 μm being preferred for most lung delivery strategies. Solid particles can also inhibit potential systemic exposure from the lung. Both liquid and solid lung delivery modes can be readily optimized in animal models such as the mouse model of bleomycin-induced fibrosis. Drug levels in the lung tissue and in the bloodstream can be readily optimized using standard assays such as ELISA.

The compositions of the invention can be administered in a variety of unit dosage forms depending on the method of administration. For example, unit dosage forms suitable for oral administration include solid dosage forms such as powder, tablets, pills, capsules, and dragees, and liquid dosage forms, such as elixirs, syrups, and suspensions. The active ingredients can also be administered parenterally in sterile liquid dosage forms. Gelatin capsules contain the active ingredient and as inactive ingredients powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The concentration of the compositions of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The compositions of the invention can also be administered via liposomes. Liposomes include emulsions, foams, micelles, insoluble monolayers, liquid crystals, phospholipid dispersions, lamellar layers and the like. In these preparations, the composition of the invention to be delivered can be incorporated as part of a liposome, alone or in conjunction with a molecule which binds to a desired target, such as antibody, or with other therapeutic or immunogenic compositions. Thus, liposomes either filled or decorated with a desired composition of the invention of the invention can be delivered systemically, or can be directed to a tissue of interest, where the liposomes then deliver the selected therapeutic/immunogenic peptide compositions.

Liposomes for use in the invention can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. Ann. Rev. Biophys. Bioeng, 9:467 (1980), U.S. Pat. Nos. 4,235,871, 4,501,728, 4,837,028, and 5,019,369.

A liposome suspension containing a composition of the invention can be administered intravenously, locally, topically, etc. in a dose which varies according to, inter alia, the manner of administration, the composition of the invention being delivered, and the stage of the disease being treated.

For solid compositions, conventional nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more compositions of the invention of the invention, and more preferably at a concentration of 25%-75%.

For aerosol administration, the compositions of the invention are preferably supplied in finely divided form along with a surfactant and propellant. Preferred percentages of compositions of the invention are 0.01%-20% by weight, preferably 1-10%. The surfactant must, of course, be nontoxic, and preferably soluble in the propellant. Representative of such agents are the esters or partial esters of fatty acids containing from 6 to 22 carbon atoms, such as c-aproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute 0.1%-20% by weight of the composition, preferably 0.25-5%. The balance of the composition is ordinarily propellant. A carrier can also be included, as desired, as with, e.g., lecithin for intranasal delivery.

The constructs of the invention can additionally be delivered in a depot-type system, an encapsulated form, or an implant by techniques well-known in the art. Similarly, the constructs can be delivered via a pump to a tissue of interest.

Any of the foregoing formulations can be appropriate in treatments and therapies in accordance with the present invention, provided that the active agent in the formulation is not inactivated by the formulation and the formulation is physiologically compatible.

### Assays for SERPINE2 Activity and SERPINE2 Antagonists

The effect of SERPINE2 on lung fibroblasts can be assessed by incubating human lung fibroblasts in presence of SERPINE2 and assessing its affect on collagen 1A1 and α-smooth muscle actin, for example, as described herein. For example, Normal human lung fibroblast (NHLF) cells from Lonza, Product number CC-2512, can be grown in Fibroblast Growth Medium containing insulin, rhFGF-B, Gentamycin Sulfate Amphotericin-B, and fetal bovine serum (FBS).

NHLF cells can be harvested from a flask with Accutase, the enzyme is neutralized with Trypsin Neutralizing Solution, the cells are pelleted, and resuspended in Full Growth Medium, counted, and plated in Falcon 96 well plates, 8000 cells per well in 200 ul per well and incubated in 37° C., 5% CO₂ for 6 hours. 6 hours after plating, cells are serum starved by removing the full growth medium and adding 200 ul of Starvation Medium (Clonetics Fibroblast Basal Medium (FBM) from Lonza Cat. # CC-3131+0.5% BSA fraction V) to the cells and incubating 16-24 hours at 37° C., 5% CO₂.

The starvation medium is removed from the cells and 75 ul of co-treatment is added followed immediately by 75 ul of protein treatment. Co-treatment is Starvation Medium with added TGF-β1 or IL-13 at one of three doses, TGF low treatment is 0.1 ng/ml TGF-(31 (final concentration in the experiments is 0.05 ng/ml); TGF high treatment is 1.0 ng/ml TGF-β1 (final concentration in the experiments is 0.5 ng/ml); IL-13 treatment is 10 ng/ml IL-13 (final concentration in the experiments is 5 ng/ml). SERPINE2 protein treatment is 75 ul of starvation medium with added recombinant SERPINE2. The level of SERPINE2 can be from 0 ng/ml to 10,000 ng/ml. After the addition of protein treatment, cells are incubated 48 hours at 37° C., 5% CO₂.

After the 48 hour treatment the medium is removed and the cells are lysed. The levels of collagen 1A1 and α-smooth muscle actin RNA are determined. The level of RNA expression can be determined by numerous techniques known in the art, such as S1 nuclease/RNase protection, PCR, bDNA, Northern blot, etc. Controls, such as β-actin can be employed.

Lung fibroblast cells that are subjected to an elevated level of SERPINE2 can be administered an antagonist of SERPINE2 to reverse the effects of the elevated levels of SERPINE2 on these cells. For example, an antagonist of SERPINE2 (e.g. monoclonal antibody) can be added to the lung fibroblast cells and, after an incubation time of 48 hours, the levels of collagen 1A1 and α-smooth muscle actin RNA can be determined. The level of SERPINE2 antagonist can be from 1 ng/ml to 10,000 ng/ml. The RNA levels can be compared in the presence and absence of the antagonist by running parallel samples or by comparing an aliquot of the sample before addition of the antagonist with an aliquot of the sample at some time(s) (e.g., 24, 48, 72 hours) after administration of the antagonist.

The effect of an antagonist can also be assessed by incubating the antagonist with SERPINE2 and determining whether the ability of SERPINE2 to complex with and inhibit trypsin-like serine proteases, such as thrombin, trypsin, plasmin, and urokinase has been altered. See, e.g., Wagner et al., 1988.

The effects of a SERPINE2 antagonist on the synthesis of collagen 1A1 and α-smooth muscle actin can be examined in a mouse model of pulmonary fibrosis. In this model, fibrosis is induced in the lungs of mice, by the intratracheal injection of the antineoplastic drug bleomycin sulfate. Bleomycin-induced fibrosis is very similar to human idiopathic pulmonary fibrosis, as documented by studies of the changes in morphology, biochemistry and mRNA in both mice and humans with this disease (Phan, S. H. Fibrotic mechanisms in lung disease. In: Immunology of Inflammation, edited by P. A. Ward, New York: Elsevier, 1983, pp 121 162; Zhang et. al. (1994) Lab. Invest. 70: 192 202; Phan and Kunkel (1992) Exper. Lung Res. 18:29 43.)

Mice can be treated by administering bleomycin and, preferably subsequently, e.g, day 10, administering the SERPINE2 antagonist. See, e.g., Moeller et al, 2008. On days 10-21 after administration of the antagonist, the lungs of the mice can be harvested and flushed with saline to remove blood, and mRNA extracted, and the expression of collagen and α-smooth muscle actin can be assessed. The administration of a SERPINE2 antagonist can ameliorate the symptoms of fibrosis in the mouse lung.

### EXAMPLES

### Example 1

### Effect of Purified SERPINE2 Protein on RNA Expression

The effect of SERPINE2 on lung fibroblasts was assessed by incubating normal human lung fibroblast (NHLF) in fibroblast growth medium. NHLF cells were harvested. The cells were then pelleted, resuspended in growth medium, plated at 8000 cells per well in 200 ul per well, and incubated in 37° C., 5% CO₂ for 6 hours. 6 hours after plating, cells were serum starved by removing the full growth medium and adding 200 ul of Starvation Medium (Clonetics Fibroblast Basal Medium (FBM) from Lonza Cat. # CC-3131+0.5% BSA fraction V) to the cells and incubating 16-24 hours at 37° C., 5% CO₂.

The starvation medium was removed from the cells and 75 ul of co-treatment was added followed immediately by 75 ul of protein treatment. Co-treatment was Starvation Medium with added TGF-β1 or IL-13 at one of three doses, TGF low treatment was 0.1 ng/ml TGF-β1 (final concentration in the experiments was 0.05 ng/ml); TGF high treatment was 1.0 ng/ml TGF-β1 (final concentration in the experiments was 0.5 ng/ml); IL-13 treatment was 10 ng/ml IL-13 (final concentration in the experiments was 5 ng/ml). SERPINE2 protein treatment was 75 ul of starvation medium with added recombinant SERPINE2. The level of SERPINE2 was from approximately 0-5000 ng/ml. After the addition of protein treatment, cells were incubated 48 hours at 37° C., 5% CO2. Human TGF-beta 1 (240-B-010), Recombinant Human IL-13 (213-IL-025) Recombinant Human SERPINE2 (2980-PI) were obtained from R&D Systems.

After the 48 hour treatment the 150 ul of medium was removed and the cells are lysed in 100 ul of 1× lysis buffer with proteinase K. The levels of collagen 1A1, β-actin, and α-smooth muscle actin were determined using a bDNA assay (Panomics). The Panomics kit instructions for the overnight hybridization and processing of samples on filter plates were followed. In the final step the beads were resuspended in 80 ul and run on the Luminex Plate Reader.

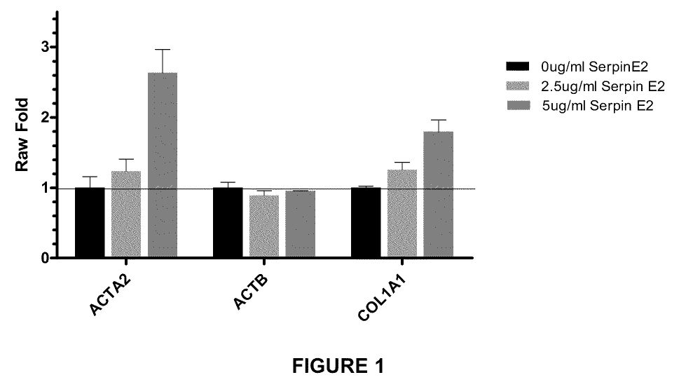

The results of an assay with purified SERPINE2 protein and 0.05 ng/ml TGF-β are shown in FIG. 1. The control RNA, β-actin, did not show any increase with SERPINE2 protein addition. However, under all three experimental conditions, the levels of collagen 1A1 and α-smooth muscle actin increased in a dose-dependent manner with increasing SERPINE2 protein. These results indicated that exposure of human lung fibroblasts to elevated levels of SERPINE2 produced an increase in both collagen 1A1, and α-smooth muscle actin expression.

### Example 2

### Generation of a Construct Expressing Wild-Type SERPINE2

A construct containing the nucleotide sequence of wild-type SERPINE2 DNA and expressing wild-type SERPINE2 protein was generated.

The nucleotide sequence of wild-type SERPINE2 DNA is:

(SEQ ID NO: 1)

atgaactggcatctccccctcttcctcttggcctctgtgacgctgccttc
catctgctcccacttcaatcctctgtctctcgaggaactaggctccaaca
cggggatccaggttttcaatcagattgtgaagtcgaggcctcatgacaac
atcgtgatctctccccatgggattgcgtcggtcctggggatgcttcagct
gggggcggacggcaggaccaagaagcagctcgccatggtgatgagatacg
gcgtaaatggagttggtaaaatattaaagaagatcaacaaggccatcgtc
tccaagaagaataaagacattgtgacagtggctaacgccgtgtttgttaa
gaatgcctctgaaattgaagtgccttttgttacaaggaacaaagatgtgt
tccagtgtgaggtccggaatgtgaactttgaggatccagcctctgcctgt
gattccatcaatgcatgggttaaaaacgaaaccagggatatgattgacaa
tctgctgtccccagatcttattgatggtgtgctcaccagactggtcctcg
tcaacgcagtgtatttcaagggtctgtggaaatcacggttccaacccgag
aacacaaagaaacgcactttcgtggcagccgacgggaaatcctatcaagt
gccaatgctggcccagctctccgtgttccggtgtgggtcgacaagtgccc
ccaatgatttatggtacaacttcattgaactgccctaccacggggaaagc
atcagcatgctgattgcactgccgactgagagctccactccgctgtctgc
catcatcccacacatcagcaccaagaccatagacagctggatgagcatca
tggtccccaagagggtgcaggtgatcctgcccaagttcacagctgtagca
caaacagatttgaaggagccgctgaaagttcttggcattactgacatgtt
tgattcatcaaaggcaaattttgcaaaaataacaaggtcagaaaacctcc
atgtttctcatatcttgcaaaaagcaaaaattgaagtcagtgaagatgga
accaaagcttcagcagcaacaactgcaattctcattgcaagatcatcgcc
tccctggtttatagtagacagaccttttctgtttttcatccgacataatc
ctacaggtgctgtgttattcatggggcagataaacaaaccc.

The amino acid sequence of wild-type SERPINE2 protein is:

(SEQ ID NO: 2)

MNWHLPLFLLASVTLPSICSHFNPLSLEELGSNTGIQVFNQIVKSRPHDN
IVISPHGIASVLGMLQLGADGRTKKQLAMVMRYGVNGVGKILKKINKAIV
SKKNKDIVTVANAVFVKNASEIEVPFVTRNKDVFQCEVRNVNFEDPASAC
DSINAWVKNETRDMIDNLLSPDLIDGVLTRLVLVNAVYFKGLWKSRFQPE
NTKKRTFVAADGKSYQVPMLAQLSVFRCGSTSAPNDLWYNFIELPYHGES
ISMLIALPTESSTPLSAIIPHISTKTIDSWMSIMVPKRVQVILPKFTAVA
QTDLKEPLKVLGITDMFDSSKANFAKITRSENLHVSHILQKAKIEVSEDG
TKASAATTAILIARSSPPWFIVDRPFLFFIRHNPTGAVLFMGQINKP.

### Example 3

### Generation of a SERPINE2 Mutein that does not Bind LRP

A construct containing the nucleotide sequence of SERPINE2 mutein that cannot bind to the low density lipoprotein receptor-related protein (LRP) was generated. This mutein contained mutations at amino acids positions 48 and 49 of SERPINE2 as follows: H48A and D49E.

The nucleotide sequence of the LRP-binding mutein of SERPINE2 DNA is:

(SEQ ID NO: 3)

atgaactggcatctccccctcttcctcttggcctctgtgacgctgccttc
catctgctcccacttcaatcctctgtctctcgaggaactaggctccaaca
cggggatccaggttttcaatcagattgtgaagtcgaggcctgcagaaaac
atcgtgatctctccccatgggattgcgtcggtcctggggatgcttcagct
gggggcggacggcaggaccaagaagcagctcgccatggtgatgagatacg
gcgtaaatggagttggtaaaatattaaagaagatcaacaaggccatcgtc
tccaagaagaataaagacattgtgacagtggctaacgccgtgtttgttaa
gaatgcctctgaaattgaagtgccttttgttacaaggaacaaagatgtgt
tccagtgtgaggtccggaatgtgaactttgaggatccagcctctgcctgt
gattccatcaatgcatgggttaaaaacgaaaccagggatatgattgacaa
tctgctgtccccagatcttattgatggtgtgctcaccagactggtcctcg
tcaacgcagtgtatttcaagggtctgtggaaatcacggttccaacccgag
aacacaaagaaacgcactttcgtggcagccgacgggaaatcctatcaagt
gccaatgctggcccagctctccgtgttccggtgtgggtcgacaagtgccc
ccaatgatttatggtacaacttcattgaactgccctaccacggggaaagc
atcagcatgctgattgcactgccgactgagagctccactccgctgtctgc
catcatcccacacatcagcaccaagaccatagacagctggatgagcatca
tggtccccaagagggtgcaggtgatcctgcccaagttcacagctgtagca
caaacagatttgaaggagccgctgaaagttcttggcattactgacatgtt
tgattcatcaaaggcaaattttgcaaaaataacaaggtcagaaaacctcc
atgtttctcatatcttgcaaaaagcaaaaattgaagtcagtgaagatgga
accaaagcttcagcagcaacaactgcaattctcattgcaagatcatcgcc
tccctggtttatagtagacagaccttttctgtttttcatccgacataatc
ctacaggtgctgtgttattcatggggcagataaacaaaccc.

The amino acid sequence of the LRP-binding mutein of SERPINE2 is:

(SEQ ID NO: 4)

MNWHLPLFLLASVTLPSICSHFNPLSLEELGSNTGIQVFNQIVKSRPAEN
IVISPHGIASVLGMLQLGADGRTKKQLAMVMRYGVNGVGKILKKINKAIV
SKKNKDIVTVANAVFVKNASEIEVPFVTRNKDVFQCEVRNVNFEDPASAC
DSINAWVKNETRDMIDNLLSPDLIDGVLTRLVLVNAVYFKGLWKSRFQPE
NTKKRTFVAADGKSYQVPMLAQLSVFRCGSTSAPNDLWYNFIELPYHGES
ISMLIALPTESSTPLSAIIPHISTKTIDSWMSIMVPKRVQVILPKFTAVA
QTDLKEPLKVLGITDMFDSSKANFAKITRSENLHVSHILQKAKIEVSEDG
TKASAATTAILIARSSPPWFIVDRPFLFFIRHNPTGAVLFMGQINKP.

### Example 4

### Generation of a SERPINE2 Mutein that can Bind to Target Proteases, but does not Irreversibly Inhibit the Proteases

A construct containing the nucleotide sequence of SERPINE2 mutein that that can bind to target proteases, but does not irreversibly inhibit the proteases was generated. This mutein (inhibition mutein) contained mutations at amino acid positions 364 and 365 of SERPINE2 as follows: R364K and S365T.

The nucleotide sequence of the SERPINE2 inhibition mutein DNA is:

(SEQ ID NO: 5)

atgaactggcatctccccctcttcctcttggcctctgtgacgctgccttc
catctgctcccacttcaatcctctgtctctcgaggaactaggctccaaca
cggggatccaggttttcaatcagattgtgaagtcgaggcctcatgacaac
atcgtgatctctccccatgggattgcgtcggtcctggggatgcttcagct
gggggcggacggcaggaccaagaagcagctcgccatggtgatgagatacg
gcgtaaatggagttggtaaaatattaaagaagatcaacaaggccatcgtc
tccaagaagaataaagacattgtgacagtggctaacgccgtgtttgttaa
gaatgcctctgaaattgaagtgccttttgttacaaggaacaaagatgtgt
tccagtgtgaggtccggaatgtgaactttgaggatccagcctctgcctgt
gattccatcaatgcatgggttaaaaacgaaaccagggatatgattgacaa
tctgctgtccccagatcttattgatggtgtgctcaccagactggtcctcg
tcaacgcagtgtatttcaagggtctgtggaaatcacggttccaacccgag
aacacaaagaaacgcactttcgtggcagccgacgggaaatcctatcaagt
gccaatgctggcccagctctccgtgttccggtgtgggtcgacaagtgccc
ccaatgatttatggtacaacttcattgaactgccctaccacggggaaagc
atcagcatgctgattgcactgccgactgagagctccactccgctgtctgc
catcatcccacacatcagcaccaagaccatagacagctggatgagcatca
tggtccccaagagggtgcaggtgatcctgcccaagttcacagctgtagca
caaacagatttgaaggagccgctgaaagttcttggcattactgacatgtt
tgattcatcaaaggcaaattttgcaaaaataacaaggtcagaaaacctcc
atgtttctcatatcttgcaaaaagcaaaaattgaagtcagtgaagatgga
accaaagcttcagcagcaacaactgcaattctcattgcaaaaacatcgcc
tccctggtttatagtagacagaccttttctgtttttcatccgacataatc
ctacaggtgctgtgttattcatggggcagataaacaaaccc.

The amino acid sequence of the SERPINE2 inhibition mutein is:

(SEQ ID NO: 6)

MNWHLPLFLLASVTLPSICSHFNPLSLEELGSNTGIQVFNQIVKSRPHDN
IVISPHGIASVLGMLQLGADGRTKKQLAMVMRYGVNGVGKILKKINKAIV
SKKNKDIVTVANAVFVKNASEIEVPFVTRNKDVFQCEVRNVNFEDPASAC
DSINAWVKNETRDMIDNLLSPDLIDGVLTRLVLVNAVYFKGLWKSRFQPE
NTKKRTFVAADGKSYQVPMLAQLSVFRCGSTSAPNDLWYNFIELPYHGES
ISMLIALPTESSTPLSAIIPHISTKTIDSWMSIMVPKRVQVILPKFTAVA
QTDLKEPLKVLGITDMFDSSKANFAKITRSENLHVSHILQKAKIEVSEDG
TKASAATTAILIAKTSPPWFIVDRPFLFFIRHNPTGAVLFMGQINKP.

### Example 5

### Generation of a SERPINE2 Mutein that Cannot Bind to Target Proteases

A construct containing the nucleotide sequence of SERPINE2 mutein that cannot bind to target proteases (interaction mutein) was generated. This mutein contained mutations at amino acid positions 364 and 365 of SERPINE2 as follows: R364P and S365P.

The nucleotide sequence of the interaction mutein of SERPINE2 DNA is:

(SEQ ID NO: 7)

atgaactggcatctccccctcttcctcttggcctctgtgacgctgccttc
catctgctcccacttcaatcctctgtctctcgaggaactaggctccaaca
cggggatccaggttttcaatcagattgtgaagtcgaggcctcatgacaac
atcgtgatctctccccatgggattgcgtcggtcctggggatgcttcagct
gggggcggacggcaggaccaagaagcagctcgccatggtgatgagatacg
gcgtaaatggagttggtaaaatattaaagaagatcaacaaggccatcgtc
tccaagaagaataaagacattgtgacagtggctaacgccgtgtttgttaa
gaatgcctctgaaattgaagtgccttttgttacaaggaacaaagatgtgt
tccagtgtgaggtccggaatgtgaactttgaggatccagcctctgcctgt
gattccatcaatgcatgggttaaaaacgaaaccagggatatgattgacaa
tctgctgtccccagatcttattgatggtgtgctcaccagactggtcctcg
tcaacgcagtgtatttcaagggtctgtggaaatcacggttccaacccgag
aacacaaagaaacgcactttcgtggcagccgacgggaaatcctatcaagt
gccaatgctggcccagctctccgtgttccggtgtgggtcgacaagtgccc
ccaatgatttatggtacaacttcattgaactgccctaccacggggaaagc
atcagcatgctgattgcactgccgactgagagctccactccgctgtctgc
catcatcccacacatcagcaccaagaccatagacagctggatgagcatca
tggtccccaagagggtgcaggtgatcctgcccaagttcacagctgtagca
caaacagatttgaaggagccgctgaaagttcttggcattactgacatgtt
tgattcatcaaaggcaaattttgcaaaaataacaaggtcagaaaacctcc
atgtttctcatatcttgcaaaaagcaaaaattgaagtcagtgaagatgga
accaaagcttcagcagcaacaactgcaattctcattgcaccaccatcgcc
tccctggtttatagtagacagaccttttctgtttttcatccgacataatc
ctacaggtgctgtgttattcatggggcagataaacaaaccc.

The amino acid sequence of the interaction mutein of SERPINE2 is:

(SEQ ID NO: 8)

MNWHLPLFLLASVTLPSICSHFNPLSLEELGSNTGIQVFNQIVKSRPHDN
IVISPHGIASVLGMLQLGADGRTKKQLAMVMRYGVNGVGKILKKINKAIV
SKKNKDIVTVANAVFVKNASEIEVPFVTRNKDVFQCEVRNVNFEDPASAC
DSINAWVKNETRDMIDNLLSPDLIDGVLTRLVLVNAVYFKGLWKSRFQPE
NTKKRTFVAADGKSYQVPMLAQLSVFRCGSTSAPNDLWYNFIELPYHGES
ISMLIALPTESSTPLSAIIPHISTKTIDSWMSIMVPKRVQVILPKFTAVA
QTDLKEPLKVLGITDMFDSSKANFAKITRSENLHVSHILQKAKIEVSEDG
TKASAATTAILIAPPSPPWFIVDRPFLFFIRHNPTGAVLFMGQINKP.

### Example 6

### Effect of SERPINE2 Muteins on Collagen 1A1 and α-Smooth Muscle Actin Expression

A control vector construct and constructs expressing wild-type SERPINE2 or SERPINE2 muteins were transfected into cells and cell supernatants were harvested.

The cDNA encoding SERPINE2 and muteins were cloned into a plasmid containing the CMV promoter for expression. The plasmid was complexed with the lipid reagent Fugene6 and transfected into human HEK293T cells plated in DMEM medium supplemented with 10% FBS and incubated at 37 in 5% CO2. After 40 hours, the cells were washed in PBS and the media is replaced with DMEM medium supplemented with 5% FBS and incubated at 37 in 5% CO2 for an additional 48 hours. The cell supernatants, containing the expressed proteins, were removed from the 293T cells and used to treat the NHLF cells in cell based assays.

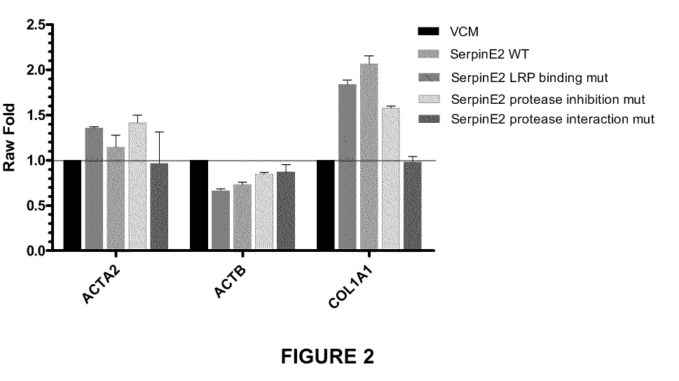

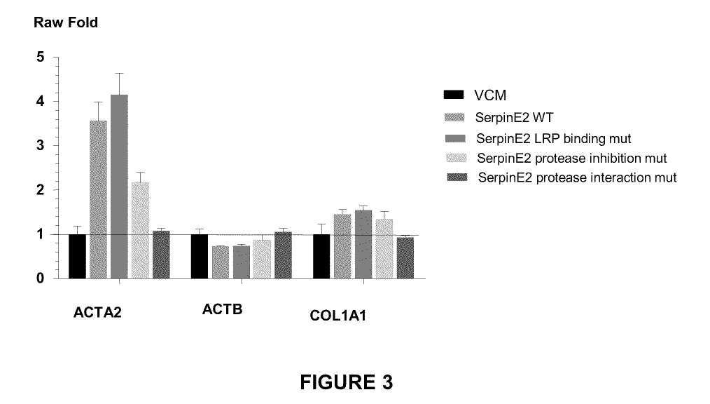

Normal human lung fibroblasts were treated with cell supernatants with 0.05 ng/ml TGF-β for 48 hours. bDNA assays were performed as in Example 1. The results are shown in FIGS. 2 and 3. The house-keeping control RNA, β-actin, did not show any increase with wild-type SERPINE2 or SERPINE2 mutein addition. However, the levels of collagen 1A1 and α-smooth muscle actin increased with addition of wild-type SERPINE2 protein. A SERPINE2 mutein having a mutation of the LRP-binding region of SERPINE2 was indistinguishable from wild-type SERPINE2. Mutation of the protease interaction region of SERPINE2 eliminated the effect. A mutant that could still bind to target proteases, but could not irreversibly inhibit them had an intermediate effect. These results indicated that the ability of SERPINE2 to inhibit its target protease is involved in the increase in collagen 1A1 and α-smooth muscle actin expression by human lung fibroblasts exposed to elevated levels of SERPINE2.

### Example 7

### SERPINE2 Induces Collagen Protein in Human Lung Fibroblasts

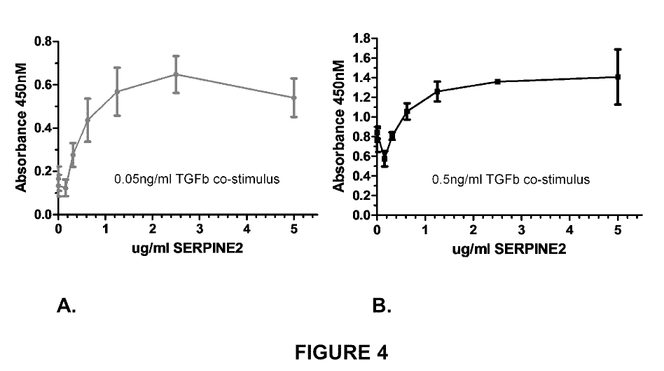

Normal human lung fibroblasts were plated 8000 cells/well of 96-well plate in 150 ul of Fibroblast Growth medium (FGM, Lonza) overnight at 37° C. Treatments (0.05 ng/ml TGF-β, 0.5 ng/ml TGF-β, and rhSerpinE2 dose curve) in 150 ul of FGM added after aspirating media the next day (time 0). Treatments in 150 ul of FGM containing 25 ug/ml ascorbic acid added after aspirating media at 24 hr. At 72 hr, cells were washed with PBS and fixed using 95% ethanol. Cells were then blocked in 1% BSA/PBS and probed using mouse anti human collagen 1 antibody #AB6308 (Abcam) at 3 ug/ml of primary antibody and goat anti mouse cat# 115-035-071 (Jackson Labs) at 1:5000 as the secondary antibody. HRP-TMB was used for detection and absorbance was read at 450 nM. The results are shown in FIG. 4. SERPINE2 was shown to have robust activity in inducing collagen protein expression in NHLF cells at both TGF-β doses.

### Example 8

### SERPINE2 Expression in Human Lung Fibroblasts

NHLF cells (Lonza) were plated at 8000 cells per well in a 96 well plate and serum starved overnight (FBM (Lonza) supplemented with 0.5% BSA (Invitrogen)) then treated with TGF-β1 (R&D Systems) in fresh starvation medium for 48 hours. RNA was extracted using a RNeasy Plus Micro kit (Qiagen). Cell lysate from 3 independent treated wells of each treatment condition were pooled for RNA isolation.

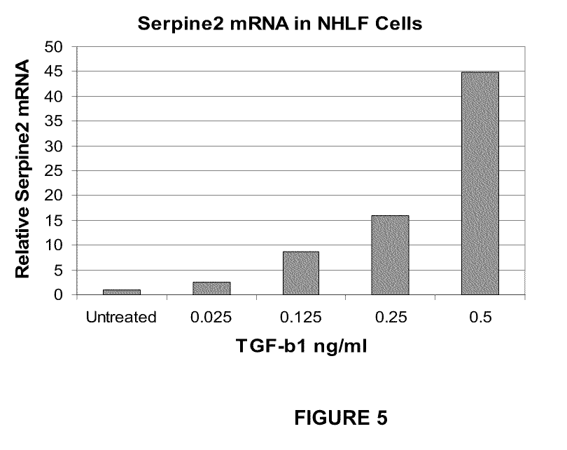

RNA was reverse transcribed using a QuantiTect Reverse Transcription kit (Qiagen) and qRT-PCR was performed using a QuantiTect SYBR Green PCR kit (Qiagen) with primers specific for human SERPINE2 (Qiagen QT00008078) and GusB (QT00046046) following manufactures protocols on an ABI 7000 instrument. SERPINE2 data was normalized to the GusB housekeeping gene using the delta-delta CT method (Applied Biosystems, Foster City, Calif.) and displayed as normalized mRNA relative to the untreated cell control. The results are shown in FIG. 5. SERPINE2 mRNA levels in NHLF cells increased dose dependently with TGF-β treatment.

### Example 9

### Inhibition of Mouse SERPINE2 Induced Collagen Production in Lung Fibroblasts using a Polyclonal Antibody to Mouse SERPINE2

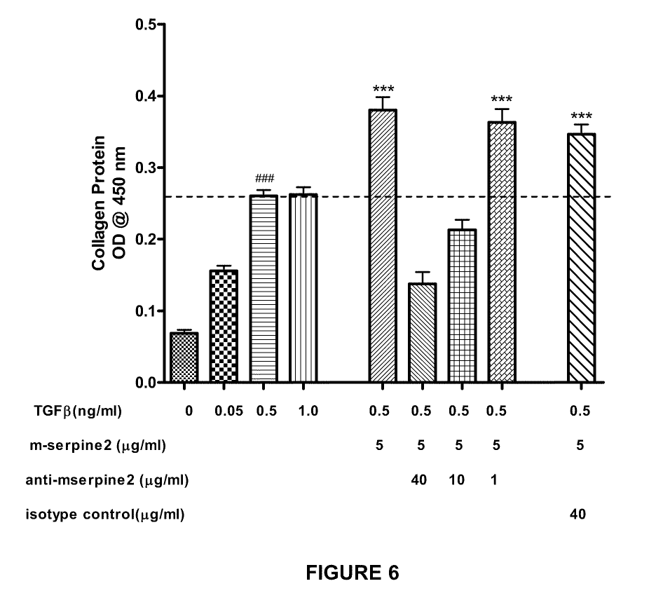

Normal human lung fibroblasts were seeded at 8000 cells per well in a 96-well tissue culture plate and allowed to attach overnight. Cells were stimulated with increasing doses of TGF-β (positive control), or TGF-β+ mouse SERPINE2. For antibody treatments, the SERPINE2 was pre-incubated with a polyclonal anti-mouse SERPINE2 antibody or an isotype control antibody for 30 minutes at room temperature, prior to addition to the cells. After 24 hours, the media was aspirated and the cells were stimulated for a further 24 hours with the above reagents in the presence of 25 μg/ml of L-ascorbic acid. After stimulation, cells were washed three times in PBS, fixed in 95% ethanol for 10 minutes at room temperature, washed again in PBS, and blocked in 1% BSA-PBS for 2 hours at room temperature. Cells were then washed thrice with PBS containing 0.1% tween-20 and incubated for 2 hours with mouse anti-human Collagen I antibody (Abcam Ab6308 1:2000, in blocking buffer). Plate was washed as before, secondary antibody (anti-mouse IgG-HRP, 1:5000 in blocking buffer) was added and incubated for 1 hour at room temperature. The plate was washed as before, and developed for 20 minutes in the dark, with TMB One solution. The assay was stopped by addition of 2N Sulfuric acid, and the optical density (OD) was read at 450 nm. The results are shown in FIG. 6.

Treatment of lung fibroblasts with TGF-β resulted in a dose-dependent increase in the amount of collagen produced. Addition of mouse SERPINE2 together with TGF-β resulted in a significant increase in collagen protein compared to TGF-β alone. As shown in the figure, pre-incubation of mouse SERPINE2 with a polyclonal anti-mouse SERPINE2 antibody completely abolished the SERPINE2-induced increase in collagen I in a dose-dependent manner, while the isotype control antibody had no effects.

### Example 10

### SERPINE2 Induction in Bleomycin Treated Mice

C57BL/6 female mice (ACE laboratories) at approximately 6 to 8 weeks of age were grouped into groups, anesthetized (isoflurane), and administered (I.T.) 40 μl of Sterile Phosphate buffered Saline (Gibco 14190) on day 0 or administered (I.T.) 40 μl of Bleomycin Sulfate (Sigma B57705: 1 U/ml in 0.9% sterile saline) on day 0.

Mice were euthanized at day 7 or day 14 by i.p. ketamine injection and used for lung tissue collection. The animals were perfused through the heart to remove blood from the lungs. Once perfused, the lung lobes from the mice were excised and flash frozen and kept in fast prep tubes until further processing.

Lung lysates were made with FastPrep Matrix D tubes in Invitrogen Cat # FNN0021 lysis buffer+Protease Inhibitor Cocktail and Phosphatase Inhibitor Cocktails 1 and 2 from Sigma. Lysates were quantified using Pierce BCA assay and boiled at 95° C. for 5 min using Biorad loading buffer (Cat# 161-0791)+BME. 2 ug total protein was loaded in each lane of a 4-12% Bis-Tris gel and run at 200V for 50 min in MOPS buffer. Transfer was carried out using the Invitrogen IBlot system. Blots were probed with 0.1 ug/ml R&D AF2175 overnight at 4° C. Subsequent to 3× washes with PBS/0.5% Tween 20, peroxidase conjugated bovine anti-goat (Jackson Cat#805-035-180) was used at 1:10,000 for 1 h at RT. Blots were then washed 6× with PBS/0.5% Tween 20 and GE Biosciences ECLPlus was used as a detection reagent. Film was developed using 30 sec, 2 minute and 4 minute exposures.

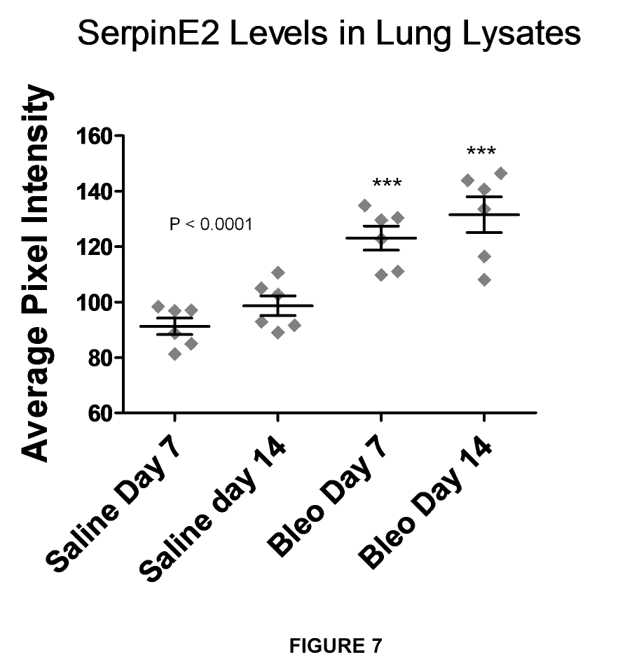

ImageJ software (http://rsb.info.nih.gov/ij/) was used to quantify average pixel intensity. Specifically, the image was inverted, and a rectangular area of fixed dimensions was placed within each band to measure average pixel intensity. Raw API values were plotted using GraphPad Prism, and statistical significance was determined using One way ANOVA with Tukey's Post test. The results are shown in FIG. 7. SERPINE2 levels (51 KD band) are significantly increased in bleo treated lung lysates as compared to saline treated.

### Example 11

### Inhibition of the Effect of SERPINE2 on Collagen 1A1 and α-Smooth Muscle Actin Expression in Human Lung Fibroblasts

A monoclonal antibody that binds to SERPINE2 and blocks its interaction with target proteases, such as thrombin, can be constructed. Wagner et al., Biochemistry 27: 2173-2176, 1988. The ability of this antibody to block the interaction of SERPINE2 with target proteases can be determined using in vitro binding assays with purified antibody and purified proteins.

The antibody can be incubated at increasing amounts with a fixed amount of SERPINE2 in the assay described in Example 1. The expression level of collagen 1A1 and α-smooth muscle actin by human lung fibroblasts can be determined using a bDNA assay. Increasing amounts of the antibody can cause a decrease in the level of expression of collagen 1A1 and α-smooth muscle actin by human lung fibroblasts.

### Example 12

### Inhibition of the Effect of SERPINE2 on Collagen 1A1 and α-Smooth Muscle Actin Expression in the Bleomycin Mouse Model

The antibody of Example 11 can be delivered via to the lungs of via aerosol at increasing amounts at various times after bleomycin treatment, for example, starting on day 12. At various times after antibody treatment, for example day 15 after bleomycin treatment, the lungs of the mice are harvested and flushed with saline to remove blood, and mRNA extracted, and the expression of collagen and α-smooth muscle actin are assessed. Increasing amounts of the antibody can cause a decrease in the level of expression of collagen 1A1 and α-smooth muscle actin by human lung fibroblasts. The administration of the antibody can ameliorate the symptoms of fibrosis in the mouse lung. The amount and timing of delivery of the antibody necessary to treat lung fibrosis in humans can be determined from these studies.

We claim:

1. A method for inhibiting the level of collagen 1A1 and/or α-smooth muscle actin expression in a human lung fibroblast cell exposed to an elevated level of SERPINE2 comprising administering an antagonist of SERPINE2 to the human lung fibroblast cell.

2. The method of claim 1, further comprising detecting a decrease in collagen 1A1 and α-smooth muscle actin expression in the lung fibroblast cell.

3. The method of claim 1, wherein the lung fibroblast cell is exposed to TGF-β prior to exposure to the antagonist.

4. The method of claim 1, wherein the lung fibroblast cell is exposed to IL-13 prior to exposure to the antagonist.

5. The method of claim 1, wherein the antagonist of SERPINE2 is an antibody.

6. The method of claim 5, wherein the antibody is a monoclonal antibody.

7. The method of claim 1, wherein the antagonist of SERPINE2 is an RNAi molecule.

8. The method of claim 1, wherein the antagonist of SERPINE2 is an antisense nucleic acid molecule.

9. The method of claim 1, wherein the antagonist of SERPINE2 is a peptide.

10. The method of claim 1, wherein the antagonist of SERPINE2 is a small molecule inhibitor of SERPINE2.

11. The method of claim 1, wherein the levels of collagen 1A1 and α-smooth muscle actin expression are inhibited.

12. The method of claim 1, wherein the level of collagen 1A1 is inhibited.

13. The method of claim 1, wherein the level of α-smooth muscle actin expression is inhibited.

14. A method for inhibiting the formation of myofibroblasts from human lung fibroblast cells exposed to an elevated level of SERPINE2 comprising administering an antagonist of SERPINE2 to the human lung fibroblast cells.

15. The method of claim 14, further comprising detecting a decrease in collagen 1A1 and α-smooth muscle actin expression in the lung fibroblast cells.

16. The method of claim 14, wherein the lung fibroblast cells are exposed to TGF-β prior to exposure to the antagonist.

17. The method of claim 14, wherein the lung fibroblast cells are exposed to IL-13 prior to exposure to the antagonist.

18. The method of claim 14, wherein the antagonist of SERPINE2 is an antibody.

19. The method of claim 17, wherein the antibody is a monoclonal antibody.

20. The method of claim 14, wherein the antagonist of SERPINE2 is an RNAi molecule.

21. The method of claim 14, wherein the antagonist of SERPINE2 is an antisense nucleic acid molecule.

22. The method of claim 14, wherein the antagonist of SERPINE2 is a peptide.

23. The method of claim 14, wherein the antagonist of SERPINE2 is a small molecule inhibitor of SERPINE2.

24. A method for increasing the level of collagen 1A1 production in a human lung fibroblast cell comprising administering SERPINE2 to a cell and detecting an increase in collagen 1A1 and α-smooth muscle actin expression in the human lung fibroblast cell.

25. The method of claim 24, wherein the SERPINE2 is administered in an expression vector.

26. The method of claim 24, wherein the SERPINE2 is administered as a purified protein.

27. The method of claim 24, wherein the increase in collagen expression is detected by measuring an increase in the level of collagen 1A1 RNA.

28. The method of claim 24, wherein the increase in α-smooth muscle actin expression is detected by measuring an increase in the level of α-smooth muscle actin RNA production.


SEQUENCE LISTING (8 sequences)

SEQ ID NO: 1 - DNA, 1191 bp, Homo sapiens
atgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaacgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtccccaagagggtgcaggtgatcctgcccaagttcacagctgtagcacaaacagatttgaaggagccgctgaaagttcttggcattactgacatgtttgattcatcaaaggcaaattttgcaaaaataacaaggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccc

SEQ ID NO: 2 - protein, 397 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITRSENLHVSHILQKAKIEVSEDGTKASAATTAILIARSSPPWFIVDRPFLFFIRHNPTGAVLFMGQINKP

SEQ ID NO: 3 - DNA, 1191 bp, Homo sapiens
atgaactggcatctccccctcttcctcttggcctctgtgacgctgccttccatctgctcccacttcaatcctctgtctctcgaggaactaggctccaacacggggatccaggttttcaatcagattgtgaagtcgaggcctgcagaaaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaacgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtccccaagagggtgcaggtgatcctgcccaagttcacagctgtagcacaaacagatttgaaggagccgctgaaagttcttggcattactgacatgtttgattcatcaaaggcaaattttgcaaaaataacaaggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccc

SEQ ID NO: 4 - protein, 397 aa, Homo sapiens
MNWHLPLFLLASVTLPSICSHFNPLSLEELGSNTGIQVFNQIVKSRPAENIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITRSENLHVSHILQKAKIEVSEDGTKASAATTAILIARSSPPWFIVDRPFLFFIRHNPTGAVLFMGQINKP

SEQ ID NO: 5 - DNA, 1191 bp, Homo sapiens
atgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaacgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtccccaagagggtgcaggtgatcctgcccaagttcacagctgtagcacaaacagatttgaaggagccgctgaaagttcttggcattactgacatgtttgattcatcaaaggcaaattttgcaaaaataacaaggtcagaaaacctccatgtttctcatatcttgcaaaaagcaaaaattgaagtcagtgaagatggaaccaaagcttcagcagcaacaactgcaattctcattgcaaaaacatcgcctccctggtttatagtagacagaccttttctgtttttcatccgacataatcctacaggtgctgtgttattcatggggcagataaacaaaccc

SEQ ID NO: 6 - protein, 397 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITRSENLHVSHILQKAKIEVSEDGTKASAATTAILIAKTSPPWFIVDRPFLFFIRHNPTGAVLFMGQINKP

SEQ ID NO: 7 - DNA, 1191 bp, Homo sapiens
atgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaacgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtccccaagagggtgcaggtgatcctgcccaagttcacagctgtagcacaaacagatttgaaggagccgctgaaagttcttggcattactgacatgtttgattcatcaaaggcaaattttgcaaaaataacaaggtcagaaaacctccatgtttctcatatcttgcaaaaagcaaaaattgaagtcagtgaagatggaaccaaagcttcagcagcaacaactgcaattctcattgcaccaccatcgcctccctggtttatagtagacagaccttttctgtttttcatccgacataatcctacaggtgctgtgttattcatggggcagataaacaaaccc

SEQ ID NO: 8 - protein, 397 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITRSENLHVSHILQKAKIEVSEDGTKASAATTAILIAPPSPPWFIVDRPFLFFIRHNPTGAVLFMGQINKP